(19) United States
(12) Patent Application Publication
(10) Pub. No.: US 2009/0023930 A1
(43) Pub. Date: Jan. 22, 2009

(54) PROCESSES AND INTERMEDIATES FOR THE PREPARATION OF HETEROCYCLIC SULFONAMIDE COMPOUNDS

(75) Inventors: Asaf Alimardanov, Nanuet, NY (US); Antonia Nikitenko, Tarrytown, NY (US); Gregg Feigelson, Chester, NY (US); John Potoski, West Nyack, NY (US)

Correspondence Address: HOWSON AND HOWSON/WYETH;CATHY A. KODROFF, SUITE 210, 501 OFFICE CENTER DRIVE, FT WASHINGTON, PA, 19034, US

(73) Assignee: Wyeth, Madison, NJ (US)

(21) Appl. No.: 12/172,472

(22) Filed: Jul. 14, 2008

Related U.S. Application Data
(60) Provisional application No. 60/959,655, filed on Jul. 16, 2007.

(51) Int. Cl. C07D 263/26; C07D 333/34; C07C 51/36

(52) U.S. Cl. 548/230; 549/65; 562/496

(57) ABSTRACT

Methods for preparing compound of formula (I) are described, wherein R₁-R₃ are defined herein, as are methods for preparing the intermediates formed therein.

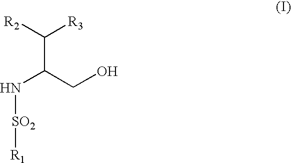

Also described are methods for enantioselectively preparing a chiral compound of the following structure, wherein R₂ and R₃ are defined herein.

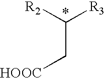

### CROSS-REFERENCE TO RELATED APPLICATIONS

This application claims the benefit of the priority of U.S. Provisional Patent Application No. 60/959,655, filed Jul. 16, 2007.

### BACKGROUND OF THE INVENTION

This invention relates to methods for preparing compounds related to beta amyloid production, including compounds which have utility in the treatment of Alzheimer's disease.

Alzheimer's disease (AD) is the most common form of dementia (loss of memory) in the elderly. The main pathological lesions of AD found in the brain consist of extracellular deposits of beta amyloid protein in the form of plaques and angiopathy and intracellular neurofibrillary tangles of aggregated hyperphosphorylated tau protein. Recent evidence has revealed that elevated beta amyloid levels in the brain not only precede tau pathology but also correlate with cognitive decline. Further suggesting a causative role for beta amyloid in AD, recent studies have shown that aggregated beta amyloid is toxic to neurons in cell culture.

Heterocyclic- and phenyl-sulfonamide compounds, specifically fluoro- and trifluoroalkyl-containing heterocyclic sulfonamide compounds, have been shown to be useful for inhibiting β-amyloid production.

What are needed in the art are alternate processes for preparing sulfonamide compounds useful for inhibiting beta amyloid production.

### SUMMARY OF THE INVENTION

In one aspect, methods for preparing sulfonamide compounds of structure (I) are described, wherein R₁-R₃ are defined herein.

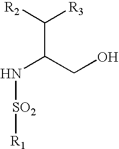

In another aspect, methods for enantioselectively preparing a chiral compound, or derivative thereof, of the following structure are described, wherein R₂ and R₃ are defined herein.

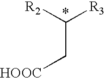

In a further aspect, the novel compounds (S)-3-(3,5-difluorophenyl)-4,4,4-trifluorobutanoic acid, (S)-4-Benzyl-3-((S)-3-(3,5-difluorophenyl)-4,4,4-trifluorobutanoyl)oxazolidin-2-one, (S)-3-((2S,3R)-2-Azido-3-(3,5-difluorophenyl)-4,4,4-trifluorobutanoyl)-4-benzyloxazolidin-2-one, (S)-3-((2S,3R)-2-Amino-3-(3,5-difluorophenyl)-4,4,4-trifluorobutanoyl)-4-benzyloxazolidin-2-one hydrochloride, (2S,3R)-2-Amino-3-(3,5-difluorophenyl)-4,4,4-trifluorobutan-1-ol hydrochloride, and N-((2S,3R)-1-((S)-4-Benzyl-2-oxo-oxazolidin-3-yl)-3-(3,5-difluorophenyl)-4,4,4-trifluoro-1-oxobutan-2-yl)-5-chlorothiophene-2-sulfonamide are provided, as are methods for independently preparing these compounds.

Other aspects and advantages of the invention will be readily apparent from the following detailed description of the invention.

### BRIEF DESCRIPTION OF THE DRAWINGS

FIG. 1. provides the powder X-ray diffraction pattern for a sample of 5-Chloro-N-((2S,3R)-3-(3,5-difluorophenyl)-4,4,4-trifluoro-1-hydroxybutan-2-yl)thiophene-2-sulfonamide prepared as described herein.

### DETAILED DESCRIPTION OF THE INVENTION

Methods are described for preparing sulfonamide compounds of structure (I). These methods are desirable over the other methods in the art since they avoid the necessity to purify the sulfonamide compounds via chromatography.

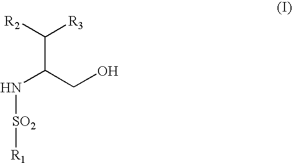

wherein, R₁ is aryl, substituted aryl, heteroaryl, or substituted heteroaryl; R₂ and R₃ are, independently, C₁ to C₆ alkyl, substituted C₁ to C₆ alkyl, aryl, substituted aryl, heteroaryl, and substituted heteroaryl. In one embodiment, R₂ and R₃ are benzyl or substituted benzyl.

The term “alkyl” is used herein to refer to both straight- and branched-chain saturated aliphatic hydrocarbon groups. In one embodiment, an alkyl group has 1 to about 10 carbon atoms (i.e., C₁, C₂, C₃, C₄, C₅ C₆, C₇, C₈, C₉, or C₁₀). In another embodiment, an alkyl group has 1 to about 6 carbon atoms (i.e., C₁, C₂, C₃, C₄, C₅ or C₆). In a further embodiment, an alkyl group has 1 to about 4 carbon atoms (i.e., C₁, C₂, C₃, or C₄).

The term “alkenyl” is used herein to refer to both straight- and branched-chain alkyl groups having one or more carbon-carbon double bonds. In one embodiment, an alkenyl group contains 2 to about 10 carbon atoms (i.e., C₂, C₃, C₄, C₅, C₆, C₇, C₈, C₉, or C₁₀). In another embodiment, an alkenyl group has 1 or 2 carbon-carbon double bonds and 2 to about 6 carbon atoms (i.e., C₂, C₃, C₄, C₅ or C₆).

The term “alkynyl” is used herein to refer to both straight- and branched-chain alkyl groups having one or more carbon-carbon triple bonds. In one embodiment, an alkynyl group has 2 to about 10 carbon atoms (i.e., C₂, C₃, C₄, C₅, C₆, C₇, C₈, C₉, or C₁₀). In another embodiment, an alkynyl group contains 1 or 2 carbon-carbon triple bonds and 2 to about 6 carbon atoms (i.e., C₂, C₃, C₄, C₅, or C₆).

The term “cycloalkyl” is used herein to refer to cyclic, saturated aliphatic hydrocarbon groups. The term cycloalkyl may include a single ring or two or more rings fused together to form a multicyclic ring structure. A cycloalkyl group may thereby include a ring system having 1 to about 5 rings. In one embodiment, a cycloalkyl group has 3 to about 22 carbon atoms (i.e., C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, or C₂₂). In another embodiment, a cycloalkyl group has 3 to about 6 carbon atoms (i.e., C₃, C₄, C₅ or C₆).

The term “substituted alkyl” refers to an group having one or more substituents including, without limitation, hydrogen, halogen, CN, OH, NO₂, amino, aryl, heterocyclic, heteroaryl, alkoxy, aryloxy, alkyloxy, alkylcarbonyl, alkylcarboxy, alkylamino, and arylthio.

The term “arylthio” as used herein refers to the S(aryl) group, where the point of attachment is through the sulfur-atom and the aryl group can be substituted as noted above.

The term “alkoxy” as used herein refers to the O(alkyl) group, where the point of attachment is through the oxygen-atom and the alkyl group can be substituted as noted above.

The term “aryloxy” as used herein refers to the O(aryl) group, where the point of attachment is through the oxygen-atom and the aryl group can be substituted as noted above.

The term “alkylcarbonyl” as used herein refers to the C(O)(alkyl) group, where the point of attachment is through the carbon-atom of the carbonyl moiety and the alkyl group can be substituted as noted above.

The term “alkylcarboxy” as used herein refers to the C(O)O(alkyl) group, where the point of attachment is through the carbon-atom of the carboxy moiety and the alkyl group can be substituted as noted above.

The term “alkylamino” as used herein refers to both secondary and tertiary amines where the point of attachment is through the nitrogen-atom and the alkyl groups can be substituted as noted above. The alkyl groups can be the same or different.

The term “halogen” as used herein refers to Cl, Br, F, or I groups.

The term “aryl” as used herein refers to an aromatic, carbocyclic system, e.g., of about 5 to 20 carbon atoms, which can include a single ring or multiple unsaturated rings fused or linked together where at least one part of the fused or linked rings forms the conjugated aromatic system. An aryl group may thereby include a ring system having 1 to about 5 rings. The aryl groups include, but are not limited to, phenyl, naphthyl, biphenyl, anthryl, tetrahydronaphthyl, phenanthryl, indene, benzonaphthyl, and fluorenyl.

The term “substituted aryl” refers to an aryl group which is substituted with one or more substituents including halogen, CN, OH, NO₂, amino, alkyl, cycloalkyl, alkenyl, alkynyl, alkoxy, C₁ to C₃ perfluoroalkyl, C₁ to C₃ perfluoroalkoxy, aryloxy, alkylcarbonyl, alkylcarboxy, —C(NH₂)═N—OH, —SO₂—(C₁ to C₁₀ alkyl), —SO₂—(C₁ to C₁₀ substituted alkyl), —O—CH₂-aryl, alkylamino, arylthio, aryl, or heteroaryl, which groups can be substituted. Desirably, a substituted aryl group is substituted with 1 to about 4 substituents.

The term “heterocycle” or “heterocyclic” as used herein can be used interchangeably to refer to a stable, saturated or partially unsaturated 3- to 20-membered monocyclic or multicyclic heterocyclic ring. The heterocyclic ring has carbon atoms and one or more heteroatoms including nitrogen, oxygen, and sulfur atoms in its backbone. In one embodiment, the heterocyclic ring has 1 to about 4 heteroatoms in the backbone of the ring. When the heterocyclic ring contains nitrogen or sulfur atoms in the backbone of the ring, the nitrogen or sulfur atoms can be oxidized. Further, when the heterocyclic ring contains nitrogen atoms, the nitrogen atoms may optionally be substituted with H, C₁ to C₆ alkyl, substituted C₁ to C₆ alkyl, CO(C₁ to C₁₂ alkyl), or CO(aryl). The heterocyclic ring can be attached through a heteroatom or carbon atom provided the resultant heterocyclic ring structure is chemically stable. When the heterocyclic ring is a multicyclic ring, it may contain 2, 3, 4, or 5 rings.

A variety of heterocyclic groups are known in the art and include, without limitation, oxygen-containing rings, nitrogen-containing rings, sulfur-containing rings, mixed heteroatom-containing rings, fused heteroatom containing rings, and combinations thereof. Examples of heterocyclic groups include, without limitation, tetrahydrofuranyl, piperidinyl, 2-oxopiperidinyl, pyrrolidinyl, morpholinyl, thiamorpholinyl, thiamorpholinyl sulfoxide, pyranyl, pyronyl, dioxinyl, piperazinyl, dithiolyl, oxathiolyl, dioxazolyl, oxathiazolyl, oxazinyl, oxathiazinyl, benzopyranyl, benzoxazinyl and xanthenyl.

The term “heteroaryl” as used herein refers to a stable, aromatic 5- to 20-membered monocyclic or multicyclic heteroatom-containing ring. The heteroaryl ring has in its backbone carbon atoms and one or more heteroatoms including nitrogen, oxygen, and sulfur atoms. In one embodiment, the heteroaryl ring contains 1 to about 4 heteroatoms in the backbone of the ring. When the heteroaryl ring contains nitrogen or sulfur atoms in the backbone of the ring, the nitrogen or sulfur atoms can be oxidized. Further, when the heteroaryl ring contains nitrogen atoms, the nitrogen atoms may optionally be substituted with H, C₁ to C₆ alkyl, substituted C₁ to C₆ alkyl, CO(C₁ to C₁₂ alkyl), or CO(aryl). The heteroaryl ring can be attached through a heteroatom or carbon atom provided the resultant heterocyclic ring structure is chemically stable. When the heteroaryl ring is a multicyclic heteroatom-containing ring, it may contain 2, 3, 4, or 5 rings.

A variety of heteroaryl groups are known in the art and include, without limitation, oxygen-containing rings, nitrogen-containing rings, sulfur-containing rings, mixed heteroatom-containing rings, fused heteroatom containing rings, and combinations thereof. Examples of heteroaryl groups include, without limitation, furyl, pyrrolyl, pyrazolyl, imidazolyl, triazolyl, pyridyl, pyridazinyl, pyrimidinyl, pyrazinyl, triazinyl, azepinyl, thienyl, dithiolyl, oxathiolyl, oxazolyl, thiazolyl, oxadiazolyl, oxatriazolyl, oxepinyl, thiepinyl, diazepinyl, benzofuranyl, thionapthene, indolyl, benzazolyl, purindinyl, pyranopyrrolyl, isoindazolyl, indoxazinyl, benzoxazolyl, quinolinyl, isoquinolinyl, benzodiazonyl, napthylridinyl, benzothienyl, pyridopyridinyl, acridinyl, carbazolyl, and purinyl rings.

The term “substituted heterocycle” and “substituted heteroaryl” as used herein refers to a heterocycle or heteroaryl group having one or more substituents including halogen, CN, OH, NO₂, amino, alkyl, cycloalkyl, alkenyl, alkynyl, C₁ to C₃ perfluoroalkyl, C₁ to C₃ perfluoroalkoxy, alkoxy, aryloxy, alkylcarbonyl, alkylcarboxy, —C(NH₂)═N—OH, —SO₂—(C₁ to C₁₀ alkyl), —SO₂—(C₁ to C₁₀ substituted alkyl), —O—CH₂-aryl, alkylamino, arylthio, aryl, or heteroaryl, may be optionally substituted. A substituted heterocycle or heteroaryl group may have 1, 2, 3, or 4 substituents.

One method for preparing the sulfonamide compounds is outlined in Scheme 1 and includes first enantioselectively hydrogenating R₂C(═CHCOOH)R₃ (compound A) to R₂CH(CH₂COOH)R₃ (compound B), wherein R₂ and R₃ are defined above. One of skill in the art would be able to purchase R₂C(═CHCOOH)R₃ for use in the method from commercial vendors. Alternatively, R₂C(═CHCOOH)R₃ may be prepared by condensing R₂C(O)R₃, which may be purchased by one of skill in the art. In one example, R₂C(O)R₃ is 1-(3,5-difluorophenyl)-2,2,2-trifluoroethanone. The condensation may be performed using conditions and reagents readily available in the art. In one embodiment, the condensation is performed using an acetic acid derivative and a base. In another embodiment, the condensing is performed using acetic anhydride and sodium acetate. See, for example, Smith, M. B.; March, J. March's Advanced Organic Chemistry, 5th edition, Wiley: NY, 2001, which is hereby incorporated by reference. Regardless of whether R₂C(═CHCOOH)R₃ is purchased or prepared from R₂C(O)R₃, it is desirably present as a single isomer. In another example, R₂C(═CHCOOH)R₃ is compound A1, wherein R₂ is defined herein.

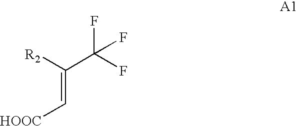

In another example, R₂C(═CHCOOH)R₃ is (E)-3-(3,5-difluorophenyl)-4,4,4-trifluoro-but-2-enoic acid (compound A2).

Asymmetric hydrogenation of compound A can be accomplished using any asymmetric hydrogenation method known to those skilled in the art. See, e.g., the hydrogenation methods described Ojima, I., ed., Catalytic Asymmetric Synthesis, 2nd edition, Wiley-VCH: New York, 2000, which is hereby incorporated by reference. In one embodiment, the hydrogenation is performed using hydrogen gas or a hydrogen transfer reagent. Desirably, the hydrogenation is performed in the presence of a catalytic or stoichiometric amount of a transition metal catalyst. The transition metal catalyst may include, without limitation, Rh, Ir, or Ru catalysts or their respective derivatives. In one embodiment, the transition metal catalyst is bis(norbornadiene)rhodium (I) tetrafluoroborate (Rh(nbd)₂BF₄). The hydrogenation is also desirably performed in the presence of a catalytic or stoichiometric amount of a chiral non-racemic compound. The term “chiral non-racemic compound” as used herein refers to a chemical compound that is present predominantly as a single enantiomer. In one embodiment, the chiral, non-racemic ligand is (R)-1-[(R)-2-(2′-dicyclohexylphosphinophenyl)-ferrocenyl]-ethyldi(bis-(3,5-trifluoromethyl)phenyl)-phosphine (Walphos 8-1). In another example, R₂CH(CH₂COOH)R₃ is compound B1, wherein R₂ is defined herein.

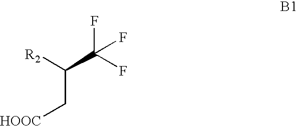

In another example, R₂CH(CH₂COOH)R₃ is (S)-3-(3,5-difluorophenyl)-4,4,4-trifluorobutanoic acid (compound B2). Desirably, compound B2 is prepared by hydrogenating compound A2 using a transition metal catalyst containing chiral non-racemic ligands and hydrogen. More desirably, compound B2 is prepared by hydrogenating compound A2 using bis(norbornadiene)rhodium (I) tetrafluoroborate, (R)-1-[(R)-2-(2′-dicyclohexylphosphinophenyl)-ferrocenyl]ethyldi(bis-(3,5-trifluoromethyl)phenyl)-phosphine, or a combination thereof.

Desirably, R₂CH(CH₂COOH)R₃ is formed from the reaction mixture at greater than 95%, 96%, 97%, 98%, or 99% enantiomeric excess. More desirably, R₂CH(CH₂COOH)R₃ is formed in greater than 99% enantiomeric excess.

Compound B is then converted to an imide of a chiral oxazolidinone (compound C), wherein R₂ and R₃ are defined herein.

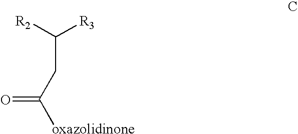

In a further example, the imide of a chiral oxazolidinone is compound C1.

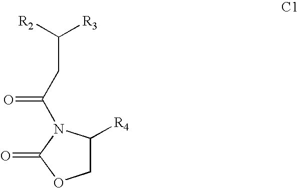

wherein, R₂ and R₃ are defined herein and R₄ is C₁ to C₆ alkyl, substituted C₁ to C₆ alkyl, aryl, or substituted aryl. In one embodiment, R₄ is benzyl or substituted benzyl.

In a further example, the imide of a chiral oxazolidinone is compound C2, wherein R₂-R₄ are defined herein.

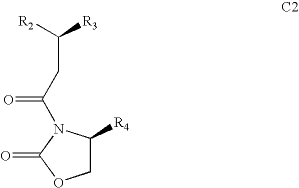

In still a further example, the imide of a chiral oxazolidinone is compound C3, wherein R₂-R₄ are defined herein.

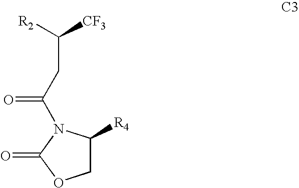

In yet another example, the imide of a chiral oxazolidinone is (S)-4-Benzyl-3-((S)-3-(3,5-difluorophenyl)-4,4,4-trifluorobutanoyl)oxazolidin-2-one (compound C4).

Compound B may then be converted directly or indirectly to compound C. In one embodiment, compound B is first converted to an acid chloride intermediate, which is thereby converted to compound C. The acid chloride may be prepared from compound B using reagents known to those of skill in the art including, without limitation, oxalyl chloride, phosphorus pentachloride, or thionyl chloride and those provided in Larock, R. C., Comprehensive Organic Transformations, 2nd ed., Wiley-VCH: New York, 1999, which is hereby incorporated by reference, optionally in the presence of a catalyst, including, without limitation, dimethylformamide. Other catalyst may be selected by one skilled in the art include those provided in Larock et al. cited above and incorporated by reference. The acid chloride intermediate is then treated with a chiral oxazolidinone. In one embodiment, the chiral oxazolidinone is compound D:

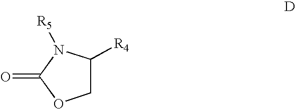

wherein, R₄ is defined above and R₅ is H, Li, Na, K, Ca, Mg, or Zn. The chiral oxazolidinone may be purchased in a form that is ready to use or may be generated in situ prior to use. In one embodiment, the chiral oxazolidinone is 4-benzyl-2-oxazolidinone, 4-phenyl-2-oxazolididone, or 4-isopropyl-2-oxazolidinone. In another embodiment, the chiral oxazolidinone is (S)-(−)-4-benzyl-2-oxazolidinone.

The chiral oxazolidinone may be prepared in situ using a variety of routes. In one route, chiral oxazolidinone D is prepared using an alkyllithium reagent and compound DD. One of skill in the art would readily be able to select a suitable alkyl lithium reagent for this purpose and may include, without limitation, n-butyllithium. Other lithium reagents may be selected by one skilled in the art and provided in Evans et al., J. Am. Chem. Soc., 1989, 111, 1063-1072, which is hereby incorporated by reference.

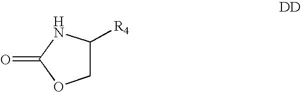

In a further route, chiral oxazolidinone D is prepared using a base and compound DD. One of skill in the art would readily be able to select a suitable base for use in this route. Suitable bases include, without limitation, 4-dimethylaminopyridine (DMAP) or triethylamine. Other bases may also be utilized and include those provided in Prashad, et al., Tet. Lett., 1998, 39, 9369-9372 and Ager et al. Synthesis, 1996, 1283-1285, which are hereby incorporated by reference. In still another route, the chiral oxazolidinone is prepared using a Grignard reagent and compound DD. One of skill in the art would readily be able to select a suitable Grignard reagent or prepare the same using reagents available in the art. Desirably, the Grignard reagent is isopropylmagnesium chloride, among others. Other Grignard reagents may be selected by one of skill in the art including those described in Williams et al., Tet. Lett., 1995, 31, 5461-5464, which is hereby incorporated by reference.

Compound B may also first be converted to a mixed anhydride and the mixed anhydride thereby converted to the imide of a chiral oxazolidinone C. One of skill in the art would readily be able to select suitable reagents to perform this sequence of reactions and may include pivaloyl chloride. In one embodiment, the mixed anhydride is prepared using pivaloyl chloride and the mixed anhydride is converted to the imide of a chiral oxazolidinone using a base. Desirably, the base is 4-(dimethylamino)pyridine. Other bases may be selected by one skilled in art include those described in Ager et al. cited above and incorporated by reference.

In one example, compound B2 is treated with pivaloyl chloride in the presence of triethylamine to form the mixed anhydride intermediate, followed by treatment with lithium oxazolidinone derivative formed separately from (S)-(−)-4-benzyl-2-oxazolidinone and n-BuLi.

In another example, compound C4 is prepared by reacting compound B2, triethylamine, and pivaloyl chloride to form a mixed anhydride, which is thereby reacted with lithium (S)-(−)-4-benzyl-2-oxazolidinone.

In a further example, compound C4 is prepared by reacting compound B2 and oxalyl chloride to form an acid chloride, which is then reacted with (S)-(−)-4-benzyl-2-oxazolidinone or a salt thereof.

In yet another example, compound C4 is prepared by reacting compound B2, triethylamine, and pivaloyl chloride to form a mixed anhydride, which is then reacted with (S)-(−)-4-benzyl-2-oxazolidinone and a base.

In yet a further example, compound C4 is prepared by reacting compound A2 and oxalyl chloride to form a acid chloride, which is then reacted with lithium (S)-(−)-4-benzyl-2-oxazolidinone to form the intermediate, which is hydrogenated using transition metal catalyst, such as Pd/C, in the presence of a Lewis acid. A variety of Lewis acids may be utilized in this reaction and include, without limitation, lithium chloride, magnesium chloride or magnesium bromide. Other Lewis acids may be selected by one of skill in the art and include those described in Smith et al. cited above and incorporated by reference.

The α-carbon atom of compound C is then substituted with an azide to form compound E, wherein R₂ and R₃ are defined herein.

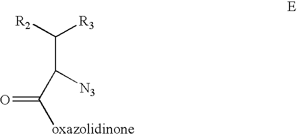

In another example, compound E1 is prepared via the azide substitution, wherein R₂-R₄ are defined herein.

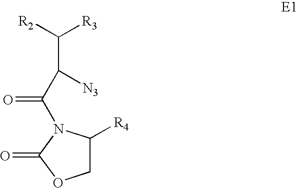

In another example, the α-carbon atom of compound C is substituted with an azide to form compound E2, wherein R₂-R₄ are defined herein.

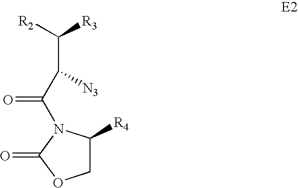

In still a further example, the α-carbon atom of compound C is substituted with an azide to form compound E3, wherein R₂-R₄ are defined herein.

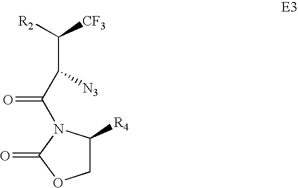

In another example, the α-carbon atom of compound C is substituted with an azide to form (S)-3-((2S,3R)-2-Azido-3-(3,5-difluorophenyl)-4,4,4-trifluorobutanoyl)-4-benzyloxazolidin-2-one (compound E4).

The introduction of the azide to the a-position of the carbonyl group of the imide of compound C is performed using any method known to those skilled in the art. In one embodiment, the reaction is performed using a second base and 2,4,6-triisopropylbenzenesulfonyl azide (trisyl azide). Desirably, an acidic quench follows. One of skill in the art would be able to select a suitable second base for use in this reaction and may include, without limitation, potassium bis(trimethylsilyl)amide, lithium bis(trimethylsilyl)amide, lithium diisopropylamide and those provided in Evans et al., J. Am. Chem. Soc., 1990, 112, 4011-4030. Desirably, the azide substitution reaction is stereoselective. In another embodiment, introduction of the azide is accomplished using potassium bis(trimethylsilyl)amide, 2,4,6-triisopropylsulphonyl azide, and acetic acid.

In one example, compound E4 is prepared by reacting compound C4, potassium bis(trimethylsilyl)amide, and 2,4,6-triisopropylsulfonylazide.

Compound E is then converted to an imide of a chiral oxazolidinone containing an amine or salt thereof (compound F, wherein R₂ and R₃ are defined herein). In one embodiment, the hydrochloride salt, hydrobromide salt, sulfuric acid salt, and acetic acid salts, among others, may be formed.

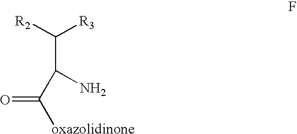

In another example, the imide of a chiral oxazolidinone containing an amine or salt thereof is compound F1, wherein R₂-R₄ are defined herein.

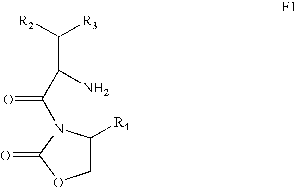

In another example, the imide of a chiral oxazolidinone containing an amine or salt thereof is compound F2, wherein R₂-R₄ are defined herein.

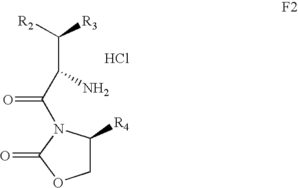

In a further example, the imide of a chiral oxazolidinone containing an amine is compound F3, wherein R₂ and R₄ are defined herein.

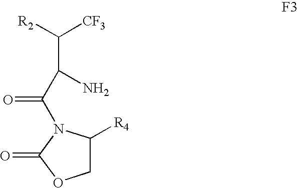

In another example, the imide of a chiral oxazolidinone containing an amine is (S)-3-((2S,3R)-2-Amino-3-(3,5-difluorophenyl)-4,4,4-trifluorobutanoyl)-4-benzyloxazolidin-2-one hydrochloride (compound F4).

The imide of a chiral oxazolidinone containing an amine or salt thereof is desirably prepared using hydrogenation. The hydrogenation conditions and reagent may be selected by those skilled in the art. In one example, the hydrogenation is performed using hydrogen, optionally in the presence of a transition metal catalyst such as Pd/C. In another example, the hydrogenation is performed using a hydrogen transfer agent, optionally in the presence of a transition metal catalyst such as Pd/C.

In one example, compound F4 is prepared by hydrogenating compound E4 in the presence of hydrochloric acid.

In a further example, the azide may be converted to the amine using P(R₁₃)₃, wherein R₁₃ is C₁ to C₁₂ alkyl, substituted C₁ to C₁₂ alkyl, aryl or substituted aryl. Suitable reagents and conditions for this transformation are provided in Larock, R. C., Comprehensive Organic Transformations, 2nd ed., Wiley-VCH: New York, 1999, which is hereby incorporated by reference.

Compound F is then reduced to aminoalcohol or salt thereof compound G, wherein R₂ and R₃ are defined herein.

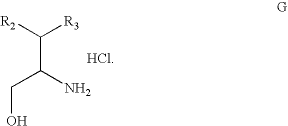

In another example, the aminoalcohol salt is compound G1, wherein R₂ and R₃ are defined herein.

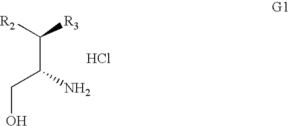

In still another example, the aminoalcohol salt is compound G2, wherein R₂ is defined herein.

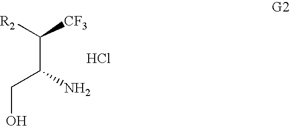

In a further example, the aminoalcohol salt is (2S,3R)-2-Amino-3-(3,5-difluorophenyl)-4,4,4-trifluorobutan-1-ol hydrochloride (compound G3). The reduction to form the aminoalcohol salt is desirably performed using a metal hydride. However, the reduction may be performed using other reducing agents available in the art including those reducing agents described in Seyden-Penne, Reductions by the Alumino- and Borohydrides in Organic Synthesis, 2nd ed., Wiley-Vch: New York, 1997, which is hereby incorporated by reference. In one example, the metal hydride is lithium borohydride or lithium aluminum hydride. In another example, the reduction is performed using lithium borohydride, followed by treatment with HCl.

In one example, compound G3 is prepared by reacting compound F4 and lithium borohydride, followed by treatment with hydrochloric acid.

In another example, compound G3 is prepared by reacting compound E4 and lithium borohydride, followed by treatment of the mixture with hydrochloric acid.

The aminoalcohol salt is then sulfonylated using techniques and reagents known to those skilled in the art including, without limitation, those described in U.S. Pat. Nos. 6,878,742; 6,610,734; and 7,166,622; US Patent Application Publication Nos. US-2005/0196813; US-2005/0171180; US-2004/0198778 and U.S. Provisional Patent Application No. 60/793,852, filed Apr. 21, 2006, which are hereby incorporated by reference herein.

The sulfonylation is desirably performed using sulfonylating reagent H or J. Desirably, the sulfonylation is performed in the presence of a base, which may readily be selected by one of skill in the art including, without limitation, 4-dimethylaminopyridine.

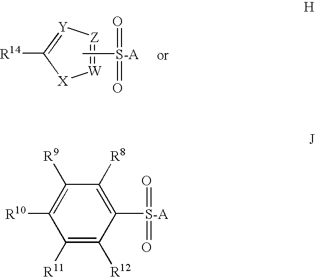

wherein, A is a leaving group; R¹⁴ is selected from among H, halogen, and CF₃; W, Y and Z are independently selected from among C, CR⁶ and N, wherein at least one of W, Y or Z is C; X is selected among O, S, SO₂, and NR⁷; R⁶ is selected from among H, halogen, C₁ to C₆ alkyl, and substituted C₁ to C₆ alkyl; R⁷ is selected from among H, C₁ to C₆ alkyl, C₃ to C₈ cycloalkyl, SO₂(C₁ to C₆ alkyl), SO₂(substituted C₁ to C₆ alkyl), SO₂aryl, SO₂substituted aryl, CO(C₁ to C₆ alkyl), CO(substituted C₁ to C₆ alkyl), COaryl and COsubstituted aryl. R⁸, R⁹, R¹⁰, R¹¹, and R¹² are independently selected from among H, halogen, C₁ to C₆ alkoxy, substituted C₁ to C₆ alkoxy, NO₂, C₁ to C₆ alkyl, substituted C₁ to C₆ alkyl, CN, C₁ to C₆ alkylcarbonyl, substituted C₁ to C₆ alkylcarbonyl, C₁ to C₆ alkylcarboxy, substituted C₁ to C₆ alkylcarboxy, CONH₂, CONH(C₁ to C₆ alkyl), CONH(substituted C₁ to C₆ alkyl), CON(C₁ to C₆ alkyl)₂, CON(substituted C₁ to C₆ alkyl)₂, S(C₁ to C₆ alkyl), S(substituted C₁ to C₆ alkyl), SO(C₁ to C₆ alkyl), SO(substituted C₁ to C₆ alkyl), SO₂(C₁ to C₆ alkyl), SO₂(substituted C₁ to C₆ alkyl), NHSO₂(C₁ to C₆ alkyl), and NHSO₂(substituted C₁ to C₆ alkyl); or R⁸ and R⁹; R⁹ and R¹⁰; R¹¹ and R¹²; or R¹⁰ and R¹¹ are fused to form (i) a saturated ring containing 3 to 8 carbon atoms; (ii) an unsaturated ring containing 5 to 8 carbon atoms; or (iii) a heterocyclic ring containing 1 to 3 heteroatoms selected from among O, N, and S in the backbone of the ring, wherein rings (i) to (iii) may be substituted by 1 to 3 substituents including C₁ to C₆ alkyl, substituted C₁ to C₆ alkyl, halogen, or CN. The term “leaving group” as used herein refers to a chemical moiety that is easily displaced from a chemical compound. Desirably, LG is Cl, Br, imidazole or sulfonate. One of skill in the art would readily be able to select a suitable leaving group for use in the sulfonylation. In one embodiment, the leaving group is halogen, sulfonate, or triflate.

In one example, the sulfonylation is performed using sulfonylating reagent H1 or J1, wherein W, X, Y, Z, R⁸-R¹², and R¹⁴ are defined above. In another example, the sulfonylation is performed using 5-chlorothiopene-2-sulphonyl chloride (H2).

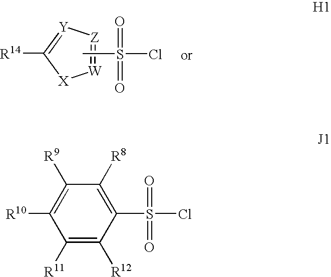

This sulfonylation step thereby provides sulfonamide compound (I).

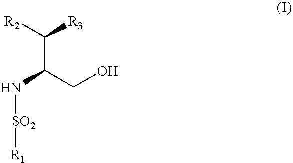

In one example, the sulfonamide compound is 5-Chloro-N-((2S,3R)-3-(3,5-fluorophenyl)-4,4,4-trifluoro-1-hydroxybutan-2-yl)thiophene-2-sulfonamide.

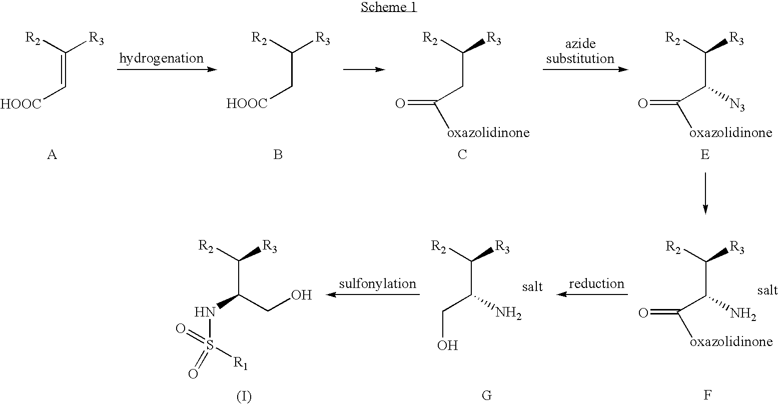

In one example, compound (Ia) is prepared as described in Scheme 2 and includes condensation of 1-(3,5-difluorophenyl)-2,2,2-trifluoroethanone to compound A2 using acetic anhydride in the presence of sodium acetate, followed by treatment with water Asymmetric hydrogenation of A2 can be accomplished using hydrogen gas in the presence of catalytic amount of bis(norbornadiene)rhodium (I) tetrafluoroborate and catalytic amount of (R)-1-[(R)-2-(2′-dicyclohexylphosphinophenyl)-ferrocenyl]-ethyldi(bis-(3,5-trifluoromethyl)phenyl)-phosphine (Walphos 8-1). The conversion of compound B2 to an imide of a chiral oxazolidinone is performed using pivaloyl chloride in the presence of triethylamine to form intermediate mixed anhydride, followed by treatment with a lithium oxazolidinone derivative formed separately from (S)-(−)-4-benzyl-2-oxazolidinone and n-BuLi. The stereoselective introduction of the azide to compound C4 is performed sequentially with potassium bis(trimethylsilyl)amide, 2,4,6-triisopropylsulphonyl azide, and acetic acid provides compound E4. Conversion of compound E4 to compound F4 is accomplished using hydrogen in the presence of Pd/C and HCl. Conversion of compound F4 to compound G3 or its hydrochloride salt is performed using LiBH₄ followed by treatment with HCl. Finally, sulfonylation with 5-chlorothiopene-2-sulphonyl chloride is performed in the presence of a base, preferably 4-dimethylaminopyridine, to form sulfonamide compound (Ia).

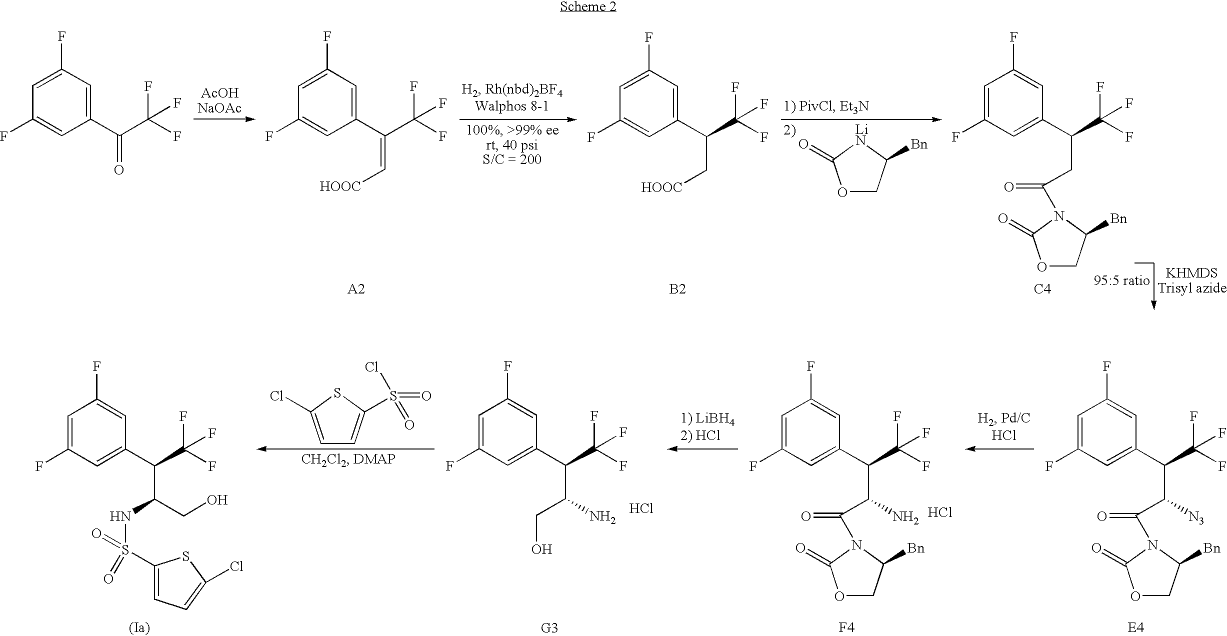

Another method for preparing sulfonamide compounds (I) is outlined in Scheme 3 and includes enantioselectively hydrogenating R₂C(═CHCOOH)R₃ (compound A) to R₂CH(CH₂COOH)R₃ (compound B) as described above. Compound B is then converted to imide of a chiral oxazolidinone C as described above. The α-carbon atom of compound C is then substituted with an azide as described above to form compound E. Compound E is then converted to an imide of a chiral oxazolidinone containing an amine or salt thereof (compound F) as described above.

Compound F is then sulfonylated using techniques and reagents known to those skilled in the art including, without limitation, those described in U.S. Pat. Nos. 6,878,742; 6,610,734; and 7,166,622; US Patent Application Publication Nos. US-2005/0196813; US-2005/0171180; US-2004/0198778 and U.S. Provisional Patent Application No. 60/793,852, filed Apr. 21, 2006, which are hereby incorporated by reference herein. The sulfonylation is performed as described above using sulfonylating reagent H or J. In another example, the sulfonylating reagent is H1 or J1. In another example, the sulfonylating reagent is 5-chlorothiopene-2-sulphonyl chloride. By doing so, sulfonylated imide K is formed, wherein R₁-R₃ are defined herein.

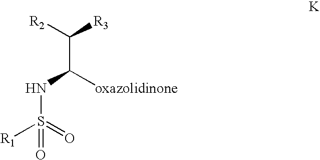

In another example, sulfonylated imide K1 is formed, wherein R₁-R₃ are defined herein.

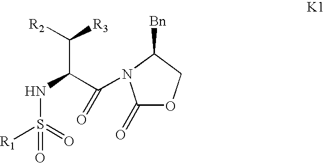

In a further example, sulfonylated imide K2 is formed, wherein R₁ and R₂ are defined herein.

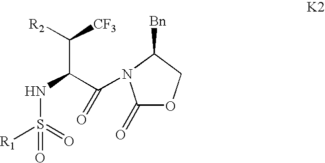

In yet a further example, sulfonylated imide N-((2S,3R)-1-((S)-4-Benzyl-2-oxo-oxazolidin-3-yl)-3-(3,5-difluorophenyl)-4,4,4-trifluoro-1-oxobutan-2-yl)-5-chlorothiophene-2-sulfonamide (compound K3) is formed.

In one example, compound K3 is prepared by reacting compound F4, a pyridine compound, and 5-chlorothiophene-2-sulfonyl chloride. The term “pyridine compound” as used herein refers to a chemical compound that contains pyridine as the backbone of the molecule and is optionally substituted by one or more substituents selected from among halogen, CN, OH, NO₂, amino, alkyl, cycloalkyl, alkenyl, alkynyl, C₁ to C₃ perfluoroalkyl, C₁ to C₃ perfluoroalkoxy, alkoxy, aryloxy, alkylcarbonyl, alkylcarboxy, —C(NH₂)═N—OH, —SO₂—(C₁ to C₁₀ alkyl), —SO₂—(C₁ to C₁₀ substituted alkyl), —O—CH₂-aryl, alkylamino, arylthio, aryl, or heteroaryl. In one embodiment, the pyridine compound is 4-(dimethylamino)pyridine. In another embodiment, the pyridine compound is pyridine.

The sulfonylated imide K may then be converted to the sulfonamide compound (I) using techniques known to those of skill in the art including reduction. A variety of reducing agents may be utilized and including, without limitation, lithium aluminum hydride or lithium borohydride, among others, including those described in Seyden-Penne, J. cited above and incorporated by reference. In one embodiment, the reduction is performed using lithium borohydride.

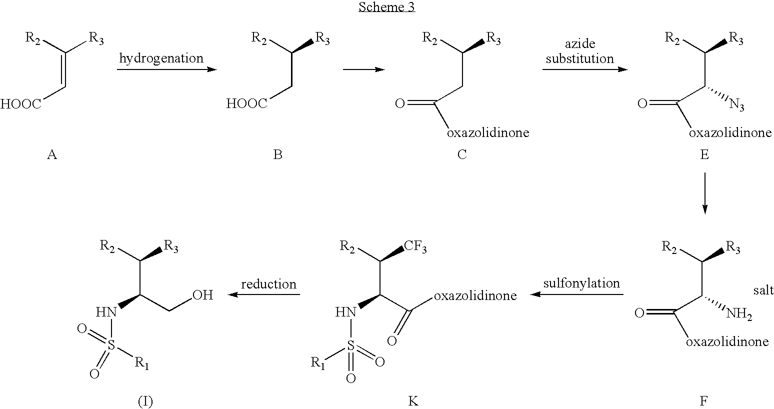

In one example, one preparation of sulfonamide compound (Ia) is described in Scheme 4 and includes condensation of 1-(3,5-difluorophenyl)-2,2,2-trifluoroethanone to compound A2 using acetic anhydride in the presence of sodium acetate, followed by treatment with water Asymmetric hydrogenation of A2 is accomplished using hydrogen gas in the presence of catalytic amount of bis(norbornadiene)rhodium (I) tetrafluoroborate and catalytic amount of (R)-1-[(R)-2-(2′-dicyclohexylphosphinophenyl)-ferrocenyl]-ethyldi(bis-(3,5-trifluoromethyl)phenyl)-phosphine (Walphos 8-1). The conversion of compound B2 to an imide of a chiral oxazolidinone C4 is performed using pivaloyl chloride in the presence of triethylamine to form intermediate mixed anhydride, followed by treatment with lithium oxazolidinone derivative formed separately from (S)-(−)-4-benzyl-2-oxazolidinone and n-BuLi. The stereoselective introduction of the azide to compound C4 is performed sequentially with potassium bis(trimethylsilyl)amide, 2,4,6-triisopropylsulphonyl azide, and acetic acid. Conversion of compound E4 to compound F4 is accomplished using hydrogen in the presence of Pd/C and HCl. Sulfonylation of compound F4 with 5-chlorothiopene-2-sulfonyl chloride is performed in the presence of 4-dimethylaminopyridine to form compound K3. Compound K3 may then be converted to the sulfonamide compound (Ia) by reaction with LiBH₄.

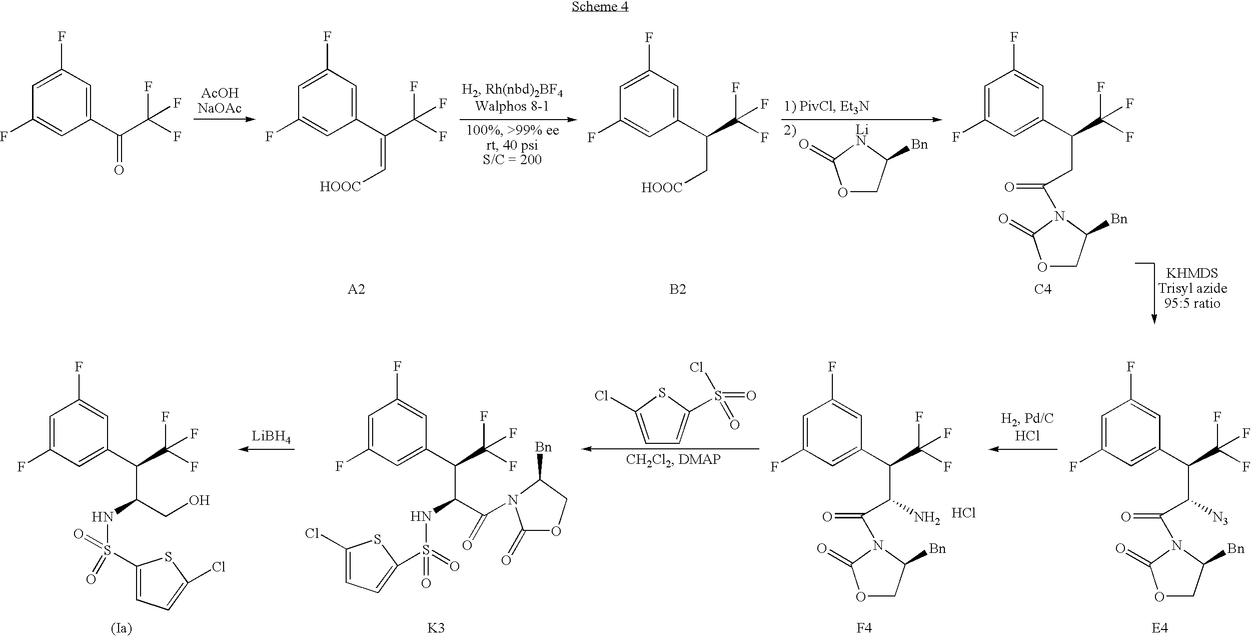

An alternative stereoselective preparation of the sulfonamide compounds (I) is provided in Scheme 5 and includes converting compound A to an unsaturated imide of a chiral oxazolidinone (compound M), wherein R₂ and R₃ are defined herein.

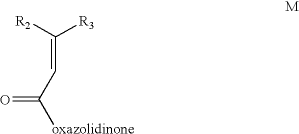

In another embodiment, an unsaturated imide of a chiral oxazolidinone M1 may be prepared, wherein R₂-R₄ are defined herein.

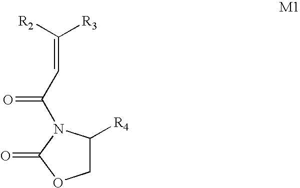

In a further embodiment, an unsaturated imide of a chiral oxazolidinone M2 may be prepared, wherein R₂-R₄ are defined herein.

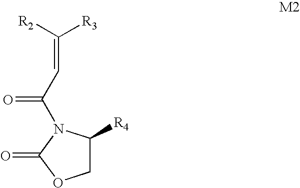

In still another embodiment, an unsaturated imide of a chiral oxazolidinone M3 may be prepared, wherein R₂ and R₄ are defined herein.

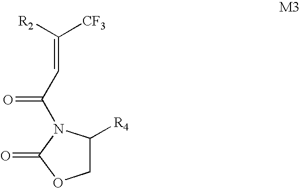

In yet a further embodiment, (S,E)-4-benzyl-3-(3-(3,5-difluorophenyl)-4,4,4-trifluorobut-2-enoyl)oxazolidin-2-one (compound M4) is prepared from compound A.

In still another embodiment, compound M5 is prepared from compound A, wherein R₂-R₄ are defined herein.

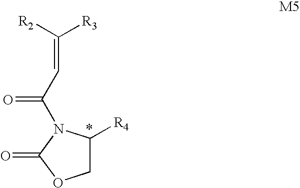

In one embodiment, conversion of A to M is performed using chiral oxazolidinone D, as described above. Desirably, the chiral oxazolidinone is 4-benzyl-2-oxazolidinone or 4-phenyl-2-oxazolididone, or 4-isopropyl-2-oxazolidinone. More desirably, the chiral oxazolidinone is (S)-(−)-4-benzyl-2-oxazolidinone. In another embodiment, conversion of A to M is performed using n-butyllithium and compound DD. In a further embodiment, conversion of A to M is performed using lithium chloride compound DD. In yet another embodiment, conversion of A to M is performed using a base and compound DD. One of skill in the art would readily be able to select a suitable base for this conversion including 4-dimethylaminopyridine or triethylamine, among others. In still another embodiment, conversion of A to M is performed using compound DD and a Grignard reagent, including those described above. In a further embodiment, conversion of A to M is performed using pivaloyl chloride and a base.

The unsaturated imide M is then diastereoselectively hydrogenated to imide C using reagents known to those of skill in the art. In one embodiment, the diastereoselective hydrogenation is performed using hydrogen or a hydrogen transfer agent, a transition metal catalyst, and a Lewis acid. A variety of Lewis acids may be utilized in the hydrogenation and include, without limitation, magnesium chloride or magnesium bromide and the Lewis acids described in Smith, M. B. cited above and incorporated by reference. The α-carbon atom of compound M is then substituted with an azide group using the azidation techniques described above to prepare compound E. Compound E is then converted to amine compound F or salt thereof using the techniques described above. Reduction of compound F to an aminoalcohol G or salt thereof may is performed using the reagents and conditions recited above. Sulfonylation of compound G as described above thereby provides the sulfonamide compound (I).

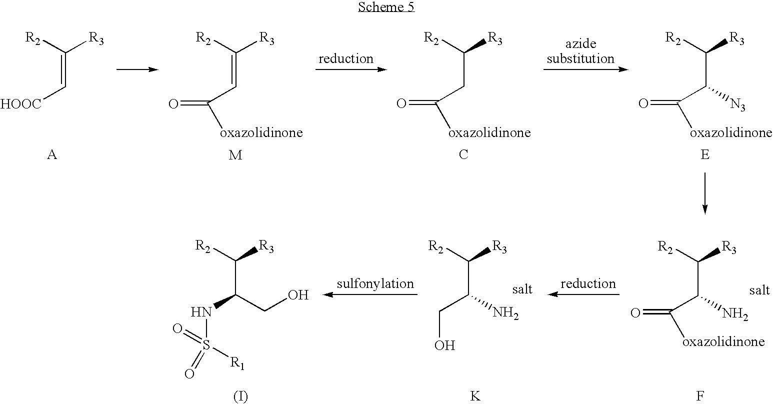

In one example, sulfonamide compound (Ia) is prepared as described in Scheme 6 by converting acid compound A2 to an unsaturated imide of a chiral oxazolidinone M4 by formation of the mixed anhydride of acid compound A2 by reaction with pivaloyl chloride in the presence of a base. The resultant intermediate is then reacted the lithium oxazolidinone derivative, which is formed separately from (S)-(−)-4-benzyl-2-oxazolidinone and n-BuLi. Hydrogenation of M4 to an unsaturated imide of a chiral oxazolidinone C4 is performed with hydrogen in the presence of a catalytic amount of 10% Pd/C and stoichiometric amount of MgBr₂. The stereoselective introduction of the azide group to compound C4 is performed sequentially with potassium bis(trimethylsilyl)amide, 2,4,6-triisopropylsulphonyl azide, and acetic acid. Conversion of compound E4 to compound F4 is accomplished using hydrogen in the presence of Pd/C and HCl. Conversion of compound F4 to compound G3 or its hydrochloride salt is performed using LiBH₄ followed by treatment with HCl. Finally, sulfonylation with 5-chlorothiopene-2-sulfonyl chloride is performed in the presence of 4-dimethylaminopyridine to form sulfonamide compound (Ia).

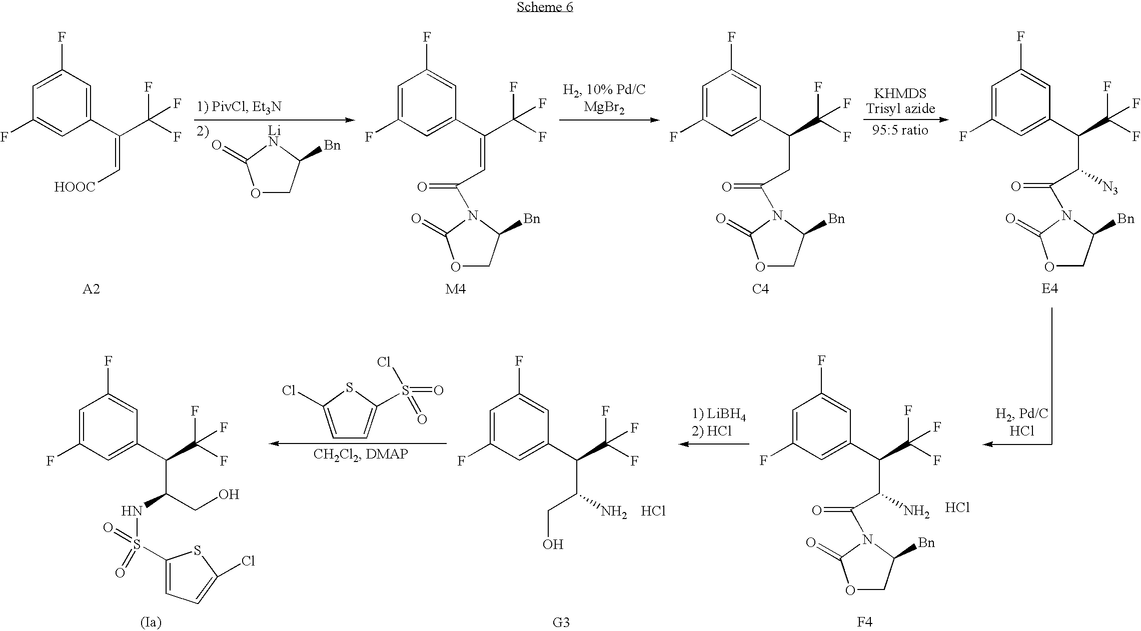

In yet another route, sulfonamide compounds (I) may be prepared as described in Scheme 7 by enantioselectively hydrogenating compound A to compound B as described above. Compound B may then be converted as described to compound C. The α-carbon atom of compound C is then substituted with an azide group using the reagents and conditions described above to provide compound E. Conversion of compound E to compound K or salt thereof is performed via reduction as described above for the reduction of compound F to compound K. Desirably, the reduction is performed using reagents and conditions known in the art including, without limitation, lithium aluminum hydride or lithium borohydride. More desirably, the reduction is performed using lithium borohydride. The reduction is typically quenched via an acid quench using the reagents and conditions described in Seyden-Penne cited above and incorporated by reference. Typically, the acid quench is performed using an acid including, without limitation, hydrochloric, hydrobromic, sulfuric, acetic, phosphoric acids. Sulfonylation of compound K using the procedures described above provides sulfonamide compounds (I).

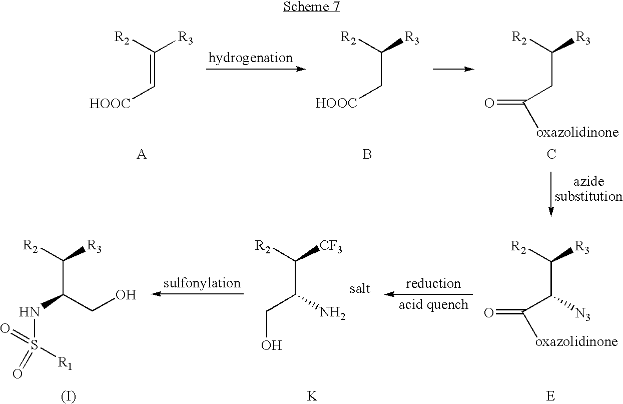

In another example, an alternative preparation of compound G3 from compound E4 is described in Scheme 8. This alternative preparation involves treatment of compound E4 with LiBH₄, followed by treatment with HCl to form compound G3.

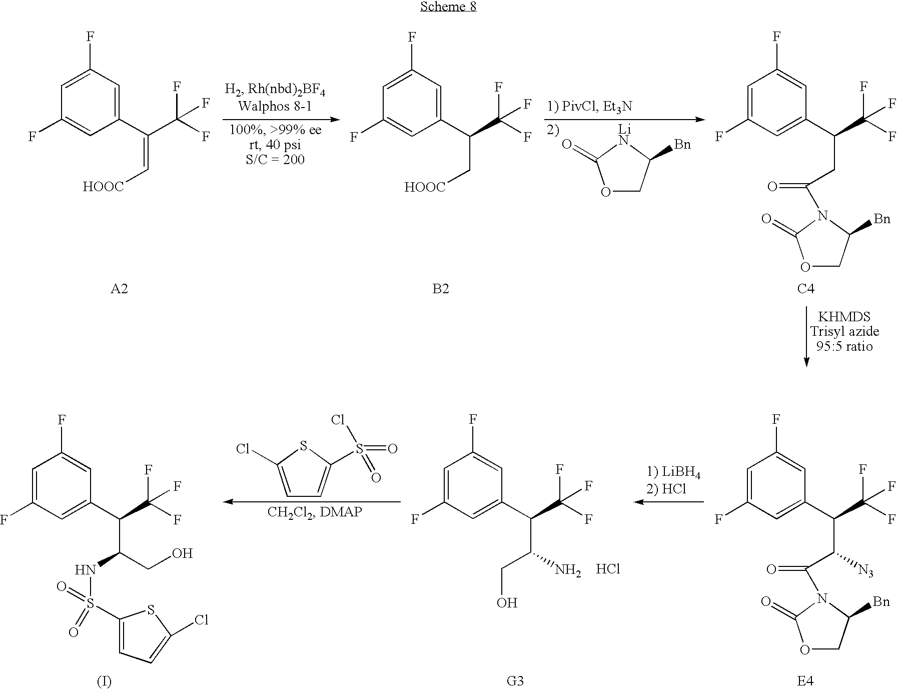

Yet another method of preparing sulfonamide compound (I) is provided in Scheme 9 and includes converting compound A to compound M as described above. Compound M is then diastereoselectively hydrogenated to compound C using the reagents and conditions provided herein. The α-carbon atom of compound C is then substituted with an azide group as described above to provide compound E. Compound E is then reduced to compound K as described herein, following by using the sulfonylation techniques discussed above to provide sulfonamide compound (I).

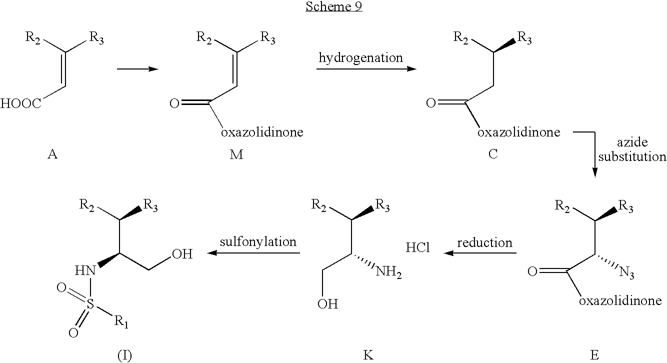

In a further example, sulfonamide compound (Ia) is prepared as described in Scheme 10 by converting acid compound A2 to an unsaturated imide of a chiral oxazolidinone M4 by formation of the mixed anhydride of acid compound A2 by reaction with pivaloyl chloride in the presence of a base. The resultant intermediate is then reacted the lithium oxazolidinone derivative, which is formed separately from (S)-(−)-4-benzyl-2-oxazolidinone and n-BuLi. Hydrogenation of M4 to an unsaturated imide of a chiral oxazolidinone C4 is performed with hydrogen in the presence of a catalytic amount of 10% Pd/C and stoichiometric amount of MgBr₂. The stereoselective introduction of the azide group to compound C4 is performed sequentially with potassium bis(trimethylsilyl)amide, 2,4,6-triisopropylsulphonyl azide, and acetic acid. Conversion of compound E4 to compound G3 hydrochloride salt is performed using LiBH₄ followed by treatment with HCl. Finally, sulfonylation with 5-chlorothiopene-2-sulfonyl chloride is performed in the presence of 4-dimethylaminopyridine to form sulfonamide compound (Ia).

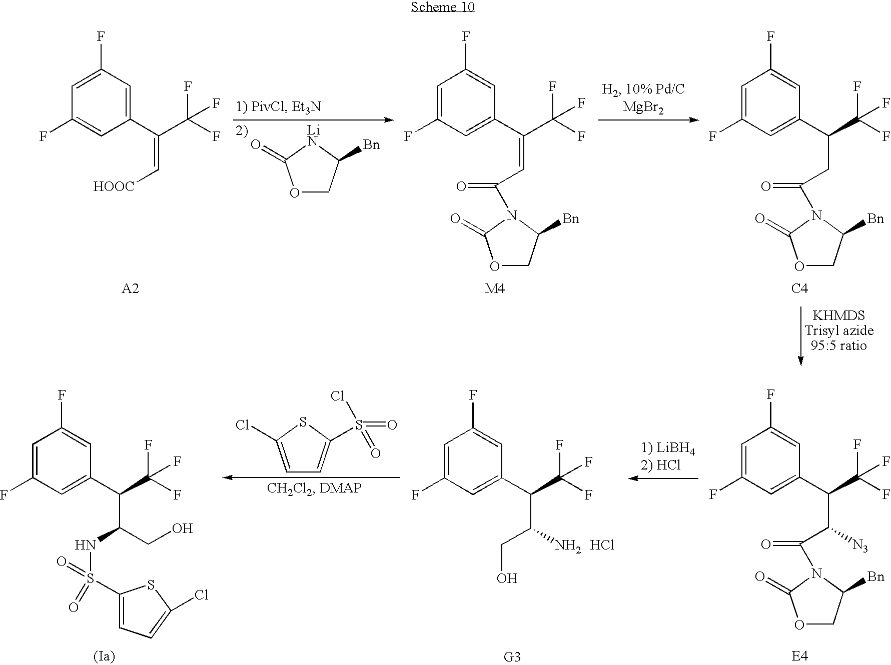

Still another method for preparing sulfonamide compounds (I) is described in Scheme 11 and includes first converting compound A to an unsaturated imide of a chiral oxazolidinone compound M using the procedures described above. Compound M is then diastereoselectively hydrogenated, as described above, to provide an imide of a chiral oxazolidinone compound C. The α-carbon atom of compound C is then substituted with an azide group to provide compound E as previously set forth. Compound E is then converted to amine compound F or salt thereof using the techniques and reagents provided herein. Sulfonylation of compound F to provide compound K, which is then reduced, each step being performed as described above, provides the sulfonamide compounds of formula (I).

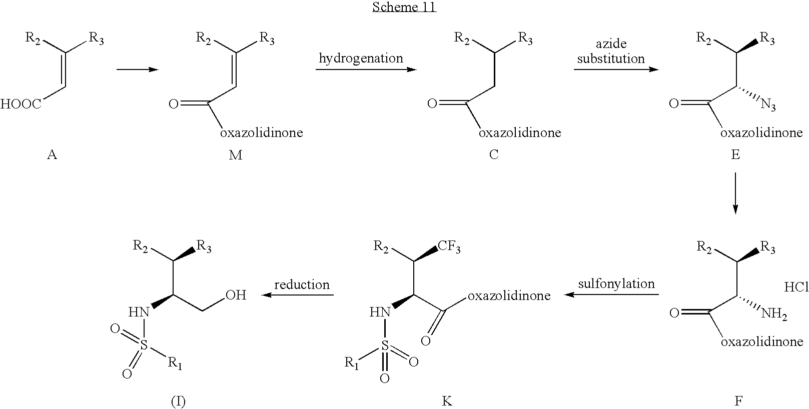

In one example, sulfonamide compound (Ia) is prepared as described in Scheme 12 by converting acid compound A2 to an unsaturated imide of a chiral oxazolidinone M4 by formation of the mixed anhydride of acid compound A2 by reaction with pivaloyl chloride in the presence of a base. The resultant intermediate is then reacted the lithium oxazolidinone derivative, which is formed separately from (S)-(−)-4-benzyl-2-oxazolidinone and n-BuLi. Hydrogenation of M4 to an unsaturated imide of a chiral oxazolidinone C4 is performed with hydrogen in the presence of a catalytic amount of 10% Pd/C and stoichiometric amount of MgBr₂. The stereoselective introduction of the azide group to compound C4 is performed sequentially with potassium bis(trimethylsilyl)amide, 2,4,6-triisopropylsulphonyl azide, and acetic acid. Conversion of compound E4 to compound F4 is accomplished using hydrogen in the presence of Pd/C and HCl. Sulfonylation of compound F4 with 5-chlorothiopene-2-sulfonyl chloride is performed in the presence of 4-dimethylaminopyridine to form compound K3. Compound K3 may then be converted to the sulfonamide compound (Ia) by reaction with LiBH₄.

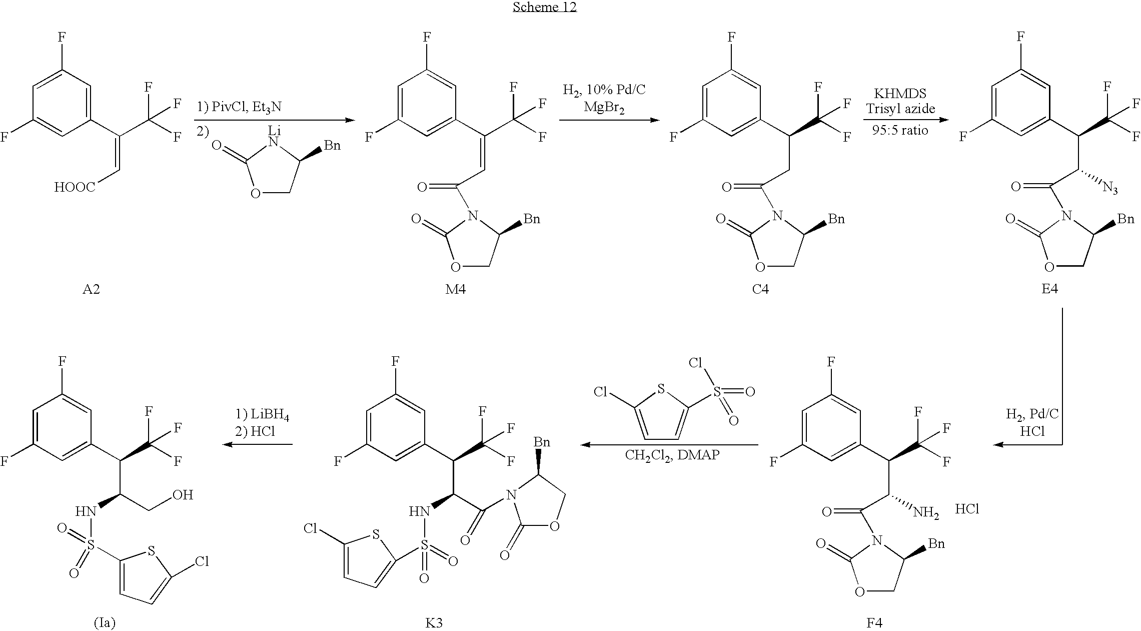

The above-noted transformations also provide enantioselective methods for preparing chiral, non-racemic compounds B*, or derivatives thereof, wherein * denotes a chiral center in enantiomerically enriched compound and R₂ and R₃. See, Scheme 13. The phrase “enantiomerically enriched compound” as used herein refers to a chemical compound that contains more that 50% of one enantiomer, desirably at least 55%, 60%, 65%, 70%, 75%, 80%, 85%, 90%, 91%, 92%, 93%, 94%, 95%, 96%, 97%, 98%, or 99% of one enantiomer of the chemical compound. Most desirably, an enantiomerically enriched compound contains 100% of a single enantiomer. The derivatives of these compounds may be prepared using the acid and methods and skill in the art. Among those derivatives that may be prepared including, without limitation, esters and amide.

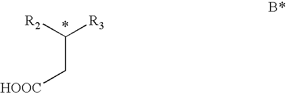

In one embodiment, the chiral, non-racemic compound is compound B*1.

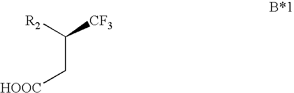

In another embodiment, the chiral, non-racemic compound is compound B*2.

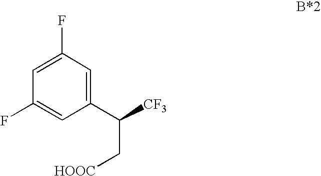

The method includes converting acid A to chiral oxazolidinone M using a chiral, non-racemic substituted oxazolidinone D and utilizing any method known to those skilled in the art, including those described above. Compound M is then hydrogenated to compound C using the hydrogenation procedures discussed above. In one embodiment, the hydrogenation is performed using hydrogen gas or hydrogen transfer agent in the presence of a transition metal, such as Pd, Pt, etc., and a Lewis acid as described above. Diastereomerically enriched compound C is then converted to the enantiomerically enriched acid B* using methods known to those skilled in the art, including those described above.

In one embodiment, acid A is converted to chiral oxazolidinone M5 using chiral, non-racemic substituted oxazolidinone D. Compound M5 is then hydrogenated to compound C2 using the hydrogenation procedures discussed above. Diastereomerically enriched compound C2 is then converted to the enantiomerically enriched acid B* using methods known to those skilled in the art, including those described above.

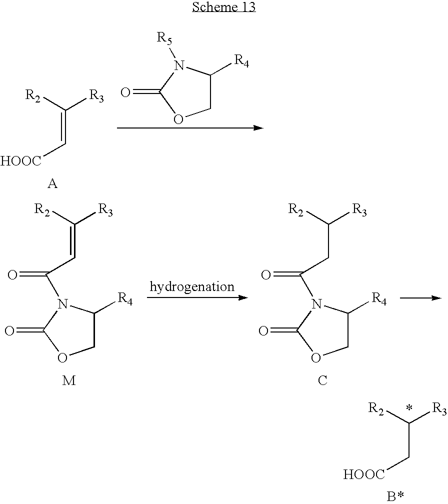

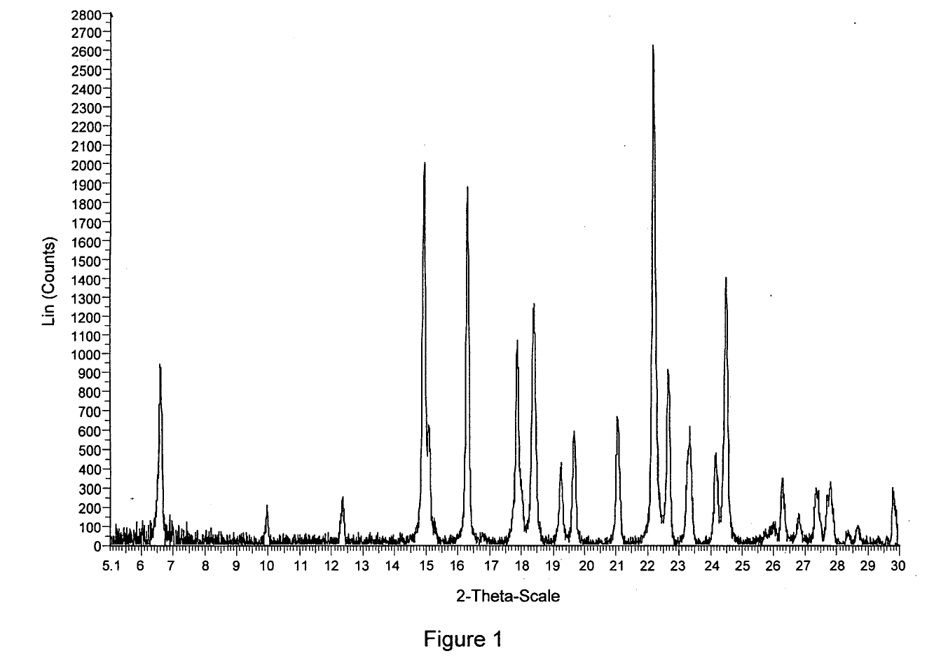

The powder XRD pattern of 5-Chloro-N-((2S,3R)-3-(3,5-difluorophenyl)-4,4,4-trifluoro-1-hydroxybutan-2-yl)thiophene-2-sulfonamide prepared as described herein was obtained using X-ray crystallographic techniques known to those of skill in the art. See, FIG. 1. In one embodiment, the XRD pattern of 5-Chloro-N-((2S,3R)-3-(3,5-difluorophenyl)-4,4,4-trifluoro-1-hydroxybutan-2-yl)-thiophene-2-sulfonamide contains one large peak and several smaller peaks. The XRD for 5-Chloro-N-((2S,3R)-3-(3,5-difluorophenyl)-4,4,4-trifluoro-1-hydroxybutan-2-yl)-thiophene-2-sulfonamide includes a peak at 2θ of about 6.5°±0.3°. The XRD for 5-Chloro-N-((2S,3R)-3-(3,5-difluorophenyl)-4,4,4-trifluoro-1-hydroxybutan-2-yl)thiophene-2-sulfonamide may also include peaks at 2θ of about 14.9°±0.3°, 22.1°±0.3°, 18.3°±0.3°, 19.6°±0.3°, 24.4°±0.3°, or 26.2°±0.3°. One of skill in the art would readily recognize that the intensities of the peaks of the powder X-ray diffraction pattern may vary. In one embodiment, the intensities of one or more peaks of the powder X-ray diffraction pattern may vary due to crystal shape, crystal size, among others.

The following examples are illustrative only and are not intended to be a limitation on the present invention.

### EXAMPLES

### Example 1

### 5-Chloro-N-((2S,3R)-3-(3,5-difluorophenyl)-4,4,4-trifluoro-1-hydroxybutan-2-yl)thiophene-2-sulfonamide

### A. (E)-3-(3,5-Difluorophenyl)-4,4,4-trifluorobut-2-enoic acid

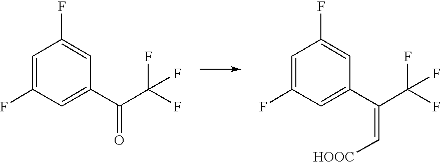

A mixture of 1-(3,5-difluorophenyl)-2,2,2-trifluoroethanone (114.59 g, 0.545 mol), NaOAc (89.48 g, 1.091 mol, 2 equiv.), and acetic anhydride (773 mL, 8.18 mol, 15 equiv.) was stirred at 100° C. for 5 hours. The reaction mixture was cooled to 4° C. and water (1.375 L) was added over 30 minutes (exotherm, 4 to 12° C.). The mixture warmed to room temperature and stirred for 2 hours (completion of hydrolysis was monitored by HPLC). Methyl t-butylether (MTBE; 1 L) was added, followed by water (1.145 L). The phases were separated and the aqueous phase was extracted with MTBE (2×0.5 L). The combined organic fraction was concentrated in vacuum and the residual AcOH and solvent were chased with toluene. 138.83 g of crude product was obtained as a yellow solid (96:4 mixture of E/Z isomers based on ¹H, ¹⁹F NMR).

The solids were dissolved in a 4:1 heptane-toluene mixture (2.7 L) at 70° C. The solution cooled to room temperature, then stirred at 2 to 4° C. for 6 hours. The precipitated solids were filtered, washed with heptane (3×150 mL), and dried in vacuum at room temperature for 24 hours to afford 117.5 g of the title product as a white solid (85% yield; single isomer as judged by ¹H, ¹⁹F NMR, and HPLC). Mp: 129-130° C. ¹H NMR (CDCl₃, δ, ppm): 6.95-6.78 (m, 3 H), 6.64 (br. q, J=1.2 Hz, 1 H). ¹⁹F NMR (CDCl₃, δ, ppm): −68.18 (s, 3 F), −109.14 (s, 2 F). ¹³C NMR (CDCl₃, δ, ppm): 168.0, 162.63 (dd, J=250, 12 Hz), 142.51 (q, J=31 Hz), 133.01 (dd, J=10, 10 Hz), 124.44 (q, J=5.4 Hz), 121.71 (q, J=274 Hz), 112.03 (dd, J=19, 8Hz), 105.31 (dd, J=25, 25 Hz). HRMS (for M−H): calc.: 251.0137; found: 251.0135.

### B. (S)-3-(3,5-Difluorophenyl)-4,4,4-trifluorobutanoic acid

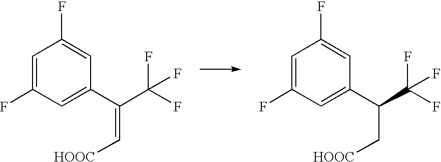

Rh(nbd)₂BF₄ (0.501 g, 0.00134 mol, 0.005 equiv), (R)-1-[(R)-2-(2′-dicyclohexylphosphinophenyl)-ferrocenyl]-ethyldi(bis-(3,5-trifluoromethyl)phenyl)-phosphine (walphos 8-1; 1.262 g, 0.00134 mol, 0.005 equiv) were placed in a 2.5-L Parr bottle. MeOH (1 L; deoxygenated by bubbling N₂ for 3 h) was added. The mixture was kept at room temperature for 30 minutes (until solids dissolved). (E)-3-(3,5-Difluorophenyl)-4,4,4-trifluorobut-2-enoic acid (67.37 g, 0.267 mol) was added. The resultant solution was hydrogenated in a Parr shaker at 45 psi H₂ at room temperature for 23 hours (HPLC analysis of an aliquot indicated consumption of starting material). Hydrogen consumption mostly took place within 1 hour. Solvent was distilled off in vacuum to afford 69.17 g of the title product as gray solid (contains residual catalyst). A single enantiomer was observed by chiral HPLC and the product was used further without purification. Mp: 73-75° C. ¹H NMR (CDCl₃, δ, ppm): 6.92-6.76 (m, 3 H), 3.94-3.78 (m, 1 H), 3.08 (dd, J=17, 4.6 Hz), 2.89 (dd, J=17, 10 Hz). ¹⁹F NMR (CDCl₃, δ, ppm): −70.72 (s, 3 F), −108.94 (s, 2 F). MS (m/z, negative ESI, for M−H): 253. [α]²⁵_{D}+28 (c=1, MeOH).

Chiral HPLC conditions: column type: the Chiralpak® AS-H column 250*4.6 mm; mobile phase: 98% heptane/trifluoroacetic acid (TFA)/2% isopropanol; flow: 1.0 mL/min; column temperature: room temperature; resolution: 2.4; injection solvent: methanol; wavelength: 254 nm; retention time (Rt) enantiomer 1: 10.0 min; Rt enantiomer 2: 11.3 min.

### C. (S)-4-Benzyl-3-((S)-3-(3,5-difluorophenyl)-4,4,4-trifluorobutanoyl)oxazolidin-2-one

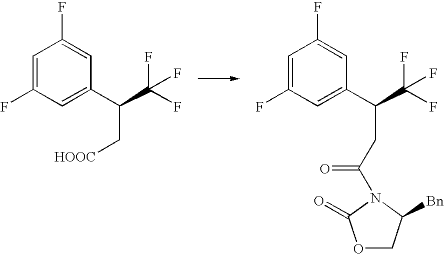

To a solution of (S)-3-(3,5-difluorophenyl)-4,4,4-trifluorobutanoic acid (50 g, 196.7 mmol) in tetrahydrofuran (THF; 250 mL) was added Et₃N (27.9 mL, 200.4 mmol, 1.02 equiv.) at −70° C., followed by trimethylacetyl chloride (24.65 mL, 200.4 mmol, 1.02 equiv). The reaction mixture warmed to −10° C., stirred at −10 to −5° C. for 2 hours, and then cooled to −70° C. (thick suspension).

In a separate flask, n-BuLi (192.5 mmol, 0.98 equiv., 77 mL of 2.5 M hexane solution) was added to a solution of (S)-(−)-4-benzyl-2-oxazolidinone (33.1 g, 186.7 mmol, 0.95 equiv.) in THF (250 mL) at −67 to −64° C. over 30 min. The mixture was stirred at −74° C. for 1 hour and then added via a cannula to the solution of mixed anhydride prepared above at −70 to −60° C. The reaction mixture was stirred at −70° C. for 1.5 hours, warmed to room temperature, and stirred for 16 hours (suspension). Water (100 mL) was added at 5° C. (solids dissolved), followed by 2N HCl (100 mL). The phases were separated and the THF phase was concentrated in vacuum. The residue was dissolved in ethylacetate (EtOAc) and the aqueous phase was extracted with MTBE. The combined organic fraction was washed with 1M Na₂CO₃ (2×200 mL), brine (200 mL), dried over MgSO₄, and concentrated to afford 79 g of crude product. The resultant solids were triturated in Et₂O (200 mL) and heptane (300 mL) was added. The solids were filtered and washed with heptane to afford 64.8 g of the title product as a white solid (80% yield based on (S)-3-(3,5-Difluorophenyl)-4,4,4-trifluorobutanoic acid). Mp: 127-128° C. ¹H NMR (CDCl₃, δ, ppm): 7.38-7.27 (m, 3 H), 7.21-7.14 (m, 2 H), 6.94 (app. d, J=6 Hz, 2 H), 6.85-6.75 (m, 1 H), 4.63-4.53 (m, 1 H), 4.18 (d, J=5 Hz, 2 H), 4.17-4.03 (m, 1 H), 3.67 (dd, J=18.5, 9.5 Hz, 1 H), 3.56 (dd, J=18.5, 4.5 Hz, 1 H), 3.26 (dd, J=13, 3.5 Hz, 1 H), 2.74 (dd, J=13, 9.5 Hz, 1 H). ¹⁹F NMR (CDCl₃, δ, ppm): −70.17 (s, 3 F), −109.14 (s, 2 F). MS (m/z, positive ESI, for M+H): 414. [α]²⁵_{D}+96.2 (c=1, MeOH).

### D. (S)-4-Benzyl-3-((S)-3-(3,5-difluorophenyl)-4,4,4-trifluorobutanoyl)oxazolidin-2-one

To a solution of (S)-3-(3,5-difluorophenyl)-4,4,4-trifluorobutanoic acid (1.06 g, 4.2 mmol) in toluene (15 mL) was added oxalyl chloride (0.63 g, 0.445 mL, 5 mmol, 1.2 equiv; 98%) at room temperature, followed by dimethylformamide (DMF; 0.01 mL). The mixture was stirred at room temperature for 1 hour. The NMR spectrum of an aliquot of this mixture indicated consumption of acid and acid chloride formation. Solvent and excess oxalyl chloride were evaporated in vacuum. Residue was dissolved in THF (12 mL).

In a separate flask, isopropyl magnesiumchloride (i-PrMgCl; 4 mmol, 2 mL of 2M THF solution) was added to a solution of (S)-(−)-4-benzyl-2-oxazolidinone (0.675 g, 3.81 mmol) in THF (12 mL) at −30° C. The mixture was stirred at −30° C. for 1.5 hours, then THF solution of acid chloride prepared as described above was added dropwise over 15 min. The reaction mixture warmed to room temperature, and stirred for 18 hours. Water (10 mL) was added. THF was distilled off in vacuum. MTBE and sodium citrate solution were added. Phases were separated, and the aqueous phase was extracted with MTBE. Combined organic fraction was washed with NaHCO₃ solution, brine, dried over MgSO₄, filtered through a pad of silica gel, and concentrated to afford 1.6 g of crude product. Recrystallization from MTBE-heptane afforded 1.3 g of the title product as a white solid (75% yield based on acid compound (IV)).

### E. (S)-3-((2S,3R)-2-Azido-3-(3,5-difluorophenyl)-4,4,4-trifluorobutanoyl)-4-benzyloxazolidin-2-one

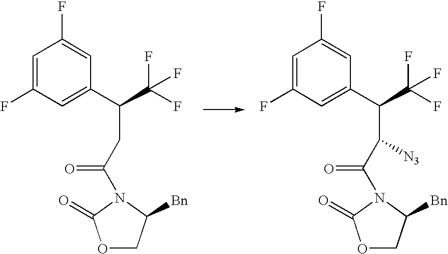

To a solution of (S)-4-benzyl-3-((S)-3-(3,5-difluorophenyl)-4,4,4-trifluorobutanoyl)-oxazolidin-2-one (65.6 g, 159 mmol) in THF (1 L), potassium hexamethyldisilazide (KHMDS; 1.1 equiv., 175 mmol, 350 mL of 0.5M toluene solution) was added at −75 to −74° C. over 50 min. The solution was stirred at −75° C. for 1 hour. A solution of 2,4,6-triisopropylsulphonyl azide (1.2 equiv, 191 mmol, 60.1 g, 97%) in THF (0.4 L) pre-cooled to −78° C. was added via a cannula at −76 to −72° C. The reaction mixture was stirred for 15 minutes, then acetic acid (4.6 equiv, 731 mmol, 42 mL) was added rapidly. The mixture warmed to room temperature. Water (500 mL) was added and the reaction mixture was kept at room temperature overnight. THF was distilled off in vacuum and EtOAc (600 mL) was added. The phases were separated and the organic phase was washed with 0.5 N HCl (2×400 mL), NaHCO₃ solution (3×400 mL), then 1M K₂CO₃ solution, brine, dried over MgSO₄, and concentrated to afford 87 g of crude product mixture. MS (m/z, positive ESI, for M+Na): 477. [α]²⁵_{D}+135 (c=1, MeOH).

### F. (S)-3-((2S,3R)-2-Amino-3-(3,5-difluorophenyl)-4,4,4-trifluorobutanoyl)-4-benzyloxazolidin-2-one hydrochloride

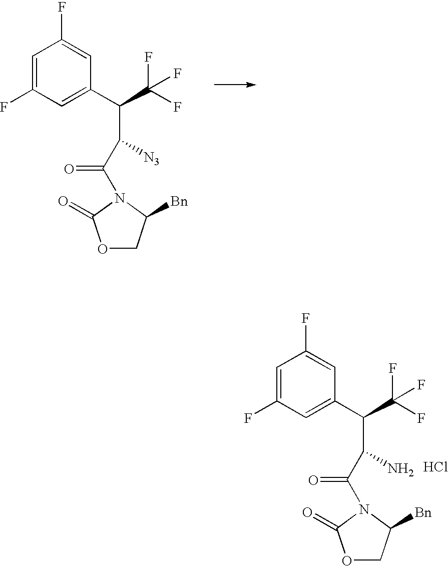

A mixture of (S)-3-((2S,3R)-2-azido-3-(3,5-difluorophenyl)-4,4,4-trifluorobutanoyl)-4-benzyloxazolidin-2-one (83.4 g of crude product from previous step), 10% Pd/C (9.6 g), MeOH (850 mL), and HCl (275 mL of 2M ether solution) was hydrogenated in a Parr shaker at 20 psi H₂ at room temperature for 1.5 hours, then at 30 psi H₂ at room temperature for 2 hours. The reaction mixture was filtered through a pad of the Celite® reagent, and concentrated to ca. 300 mL volume. Et₂O (600 mL) was added, followed by heptane (300 mL). The precipitated solids were filtered, washed with ether, and dried in vacuum at room temperature for 24 hours to afford 60 g of the title product as a white solid (85% yield after two steps). Mp: 151-153° C. ¹H NMR (CD₃OD, δ, ppm): 7.37-7.04 (m, 8 H), 5.9 (d, J=9 Hz, 1 H), 4.52 (p, J=9 Hz, 1 H), 4.4-4.3 (m, 1 H), 4.21 (dd, J=9, 2 Hz, 1 H), 3.9 (dd, J=9, 7.5 Hz, 1 H), 3.23 (dd, J=13.5, 3 Hz, 1 H), 2.85 (dd, J=13.5, 9 Hz, 1 H). ¹⁹F NMR (CD₃OD, δ, ppm): −65.79 (s, 3 F), −109.23 (s, 2 F). MS (m/z, positive ESI, for M+H): 429.

### G. (2S,3R)-2-Amino-3-(3,5-difluorophenyl)-4,4,4-trifluorobutan-1-ol hydrochloride

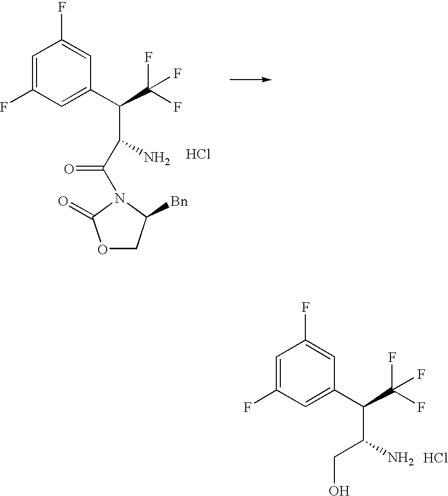

A solution of (S)-3-((2S,3R)-2-amino-3-(3,5-difluorophenyl)-4,4,4-trifluorobutanoyl)-4-benzyloxazolidin-2-one hydrochloride (55.6 g, 120 mmol) in THF (400 mL) was added to LiBH₄ solution (480 mmol, 240 mL of 2N THF solution) at 0 to 5° C. over 1.5 hours (gas evolution). Cooling bath was removed and the reaction mixture was stirred for 30 minutes while warming to room temperature. The reaction mixture was added to MeOH (1.5 L) at 0 to 5° C. over 1 hour (gas evolution). The solution was stirred for 2 hours and a 2N HCl solution (400 mL) was added at 0 to 5° C. The mixture warmed to room temperature and stirred for 16 hours (HPLC monitored for intermediate amine-borane complex decomposition). THF and MeOH were distilled off in vacuum. 2N HCl (200 mL) was added to the residue and the solution washed with CH₂Cl₂ (3×300 mL). The aqueous layer was basified with K₂CO₃ (55 g) at 15° C. and extracted with MTBE (2×300 mL). NaCl (40 g) was added to the aqueous layer and the aqueous phase was extracted with MTBE (300 mL). The combined MTBE solution was dried over K₂CO₃, filtered, and concentrated to afford 28.4 g of crude product. The crude product was dissolved in Et₂O (200 mL) and MeOH (10 mL). HCl (70 mL of 2N solution in ethyl ether) was added. The mixture was stirred at room temperature for 30 min, cooled to 0° C., filtered, washed with ether and heptane, and air dried for 1 hour to afford 29.9 g of the title product as white solid (86% yield). Mp: 231-233° C. ¹H NMR (CD₃OD, δ, ppm): 7.15-7.05 (m, 3 H), 4.14-3.96 (m, 2 H), 3.64 (dd, J=12, 2 Hz, 1 H), 3.25 (dd, J=12, 3.5 Hz, 1 H). ¹⁹F NMR (CD₃OD, δ, ppm): −67.62 (s, 3 F), −110.9 (s, 2 F). MS (m/z, positive ESI, for M+H): 256. [α]²⁵_{D}+40.4 (c=1, MeOH).

### H. 5-Chloro-N-((2S,3R)-3-(3,5-difluorophenyl)-4,4,4-trifluoro-1-hydroxybutan-2-yl)thiophene-2-sulfonamide

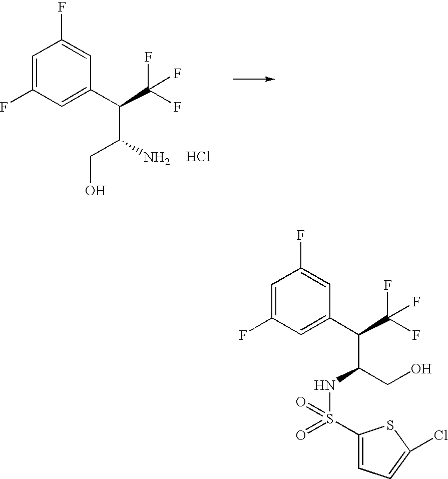

To a solution of (2S,3R)-2-amino-3-(3,5-difluorophenyl)-4,4,4-trifluorobutan-1-ol hydrochloride (29.1 g, 0.1 mol) and 4-(dimethylamino)-pyridine (DMAP, 27 g, 0.22 mol) in dichloromethane (800 mL) a solution of 5-chlorothiopene-2-sulphonyl chloride (22.3 g, 0.103 mol) in dichloromethane (60 mL) was added dropwise (mild exotherm). The mixture was stirred at room temperature for 2 hours, washed with 2N HCl (3×300 mL), brine (300 mL), NaHCO₃ solution (300 mL), dried over MgSO₄, and concentrated. The residue was dissolved in MTBE, washed with 0.5N HCl, brine, dried over MgSO₄, and concentrated to afford 42.4 g of crude product. The crude product was dissolved in Et₂O (100 mL) and heptane (500 mL) was added dropwise. The precipitated solids were filtered, washed with heptane, and dried in vacuum at room temperature to afford 32 g of the title product as off-white solid (73% yield).

### I. Purification of 5-Chloro-N-((2S,3R)-3-(3,5-difluorophenyl)-4,4,4-trifluoro-1-hydroxybutan-2-yl)thiophene-2-sulfonamide

5-Chloro-N-((2S,3R)-3-(3,5-difluorophenyl)-4,4,4-trifluoro-1-hydroxybutan-2-yl)thiophene-2-sulfonamide (59 g) was dissolved in Et₂O (300 mL) and filtered through a pad of silica gel to remove a polar impurity. The silica gel was then washed with Et₂O (200 mL) and the solution was concentrated. The residue was dissolved in Et₂O (120 mL) and heptane (1.25 L) was added dropwise over 1.5 hours. The precipitate was filtered, washed with heptane, and dried in vacuum at room temperature for 24 hours to afford 56.18 g of the title product as a white solid. Mp 125-126° C. (98.8% pure as judged by HPLC analysis at 215 nm; single isomer detected in chiral HPLC) ¹H NMR (CDCl₃, δ, ppm): 7.44 (d, J=4 Hz, 1 H), 6.94 (d, J=4 Hz, 1 H), 6.88-6.78 (m, 3 H), 5.25 (d, J=8 Hz, 1 H), 3.96-3.64 (m, 3 H), 3.3 (ddd, J=11, 4.6, 3.8 Hz, 1 H), 1.74 (t, J=4.6 Hz, 1 H). ¹⁹F NMR (CDCl₃, δ, ppm): −63.91 (s, 3 F), −108.07 (s, 2 F). Anal. calc. for C₁₄H₁₁ClF₅NO₃S₂: C, 38.58%, H, 2.54%, N, 3.21%; found: C, 38.69%, H, 2.7%, N, 3.16%. HRMS (for M+H) calc.: 435.98618; found: 435.98728. [α]²⁵_{D}+33.6 (c=1, MeOH).

Chiral HPLC conditions: column type: the Chiralcel® AD column, 250*4.6 mm; mobile phase: 15% isopropanol in hexane; flow: 1.0 mL/min; column temperature: room temperature; injection solvent: ethanol; wavelength: 254 nm; Rt isomer 1: 4.66 min; Rt isomer 2: 4.79 min; Rt isomer 3 (isomer of interest): 5.54 min; Rt isomer 4: 7.51 min.

### Example 2

### 5-Chloro-N-((2S,3R)-3-(3,5-difluorophenyl)-4,4,4-trifluoro-1-hydroxybutan-2-yl)thiophene-2-sulfonamide

### A. N-((2S,3R)-1-((S)-4-Benzyl-2-oxo-oxazolidin-3-yl)-3-(3,5-difluorophenyl)-4,4,4-trifluoro-1-oxobutan-2-yl)-5-chlorothiophene-2-sulfonamide

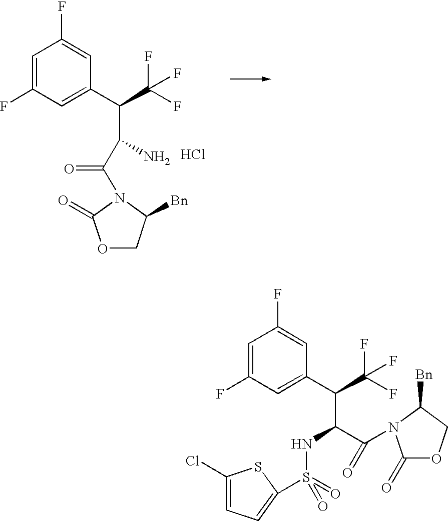

A mixture of (S)-3-((2S,3R)-2-amino-3-(3,5-difluorophenyl)-4,4,4-trifluorobutanoyl)-4-benzyloxazolidin-2-one hydrochloride (10 g, 21.6 mmol), 5-chlorothiopene-2-sulphonyl chloride (9.4 g, 43.2 mmol), DMAP (5.6 g, 43.2 mmol), pyridine (3.4 g, 3.5 mL, 43.2 mmol), and dichloromethane (300 mL) was stirred at room temperature for 40 hours. A NaHCO₃ solution (300 mL) was added. The phases were separated and the organic phase was washed with 2N HCl (2×300 mL), brine, and concentrated. Flash chromatography purification (silica gel, methylene chloride) afforded 9.3 g of the title product (71% yield). ¹H NMR (CD₃OD, δ, ppm): 7.42 (d, J=4 Hz, 1 H), 7.34-7.22 (m, 3 H), 7.20-7.15 (m, 2 H), 7.04 (d, J=4 Hz, 1 H), 7.03-6.96 (m, 3 H), 5.91 (d, J=7 Hz, 1 H), 4.38-4.30 (m, 1 H), 4.29-4.19 (m, 1 H), 4.13 (dd, J=9, 2 Hz, 1 H), 4.0 (dd, J=9, 8 Hz, 1 H), 2.93 (dd, J=13.5, 3 Hz, 1 H), 2.57 (dd, J=13.5, 9 Hz, 1 H). MS (m/z, positive ESI, for M+H): 609. MS (m/z, negative ESI, for M−H): 607.

### B. 5-Chloro-N-((2S,3 R)-3-(3,5-difluorophenyl)-4,4,4-trifluoro-1-hydroxybutan-2-yl)thiophene-2-sulfonamide

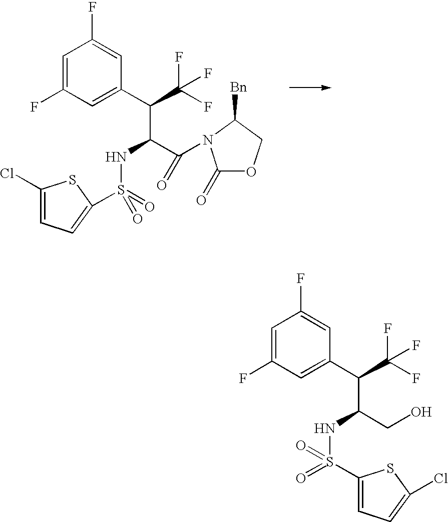

To a solution of N-((2S,3R)-1-((S)-4-benzyl-2-oxo-oxazolidin-3-yl)-3-(3,5-difluorophenyl)-4,4,4-trifluoro-1-oxobutan-2-yl)-5-chlorothiophene-2-sulfonamide (9.17 g, 15.05 mmol) in THF (300 mL) was slowly added LiBH₄ (30.1 mL of 2N THF solution, 60.2 mmol) (exotherm; gas evolution). The reaction mixture was stirred at room temperature for 2.5 hours and MeOH (45 mL) was added at 0 to 10° C. (gas evolution). Solvent was evaporated in vacuum and the residue was dissolved in dichloromethane. The solution was washed with 2N HCl, dried over MgSO₄, and concentrated to afford 5.24 g of solid. The solid was dissolved in ethyl ether (18 mL) and heptane (50 mL) was added dropwise. Precipitated solids were filtered, washed with heptane, and dried in vacuum at room temperature to afford 4.96 g of the title product as a white solid (76% yield. 96.9% pure as judged by HPLC analysis at 215 nm. 98.8% isomeric purity as determined by chiral HPLC (chiral HPLC conditions described above).) ¹H NMR (CDCl₃, δ, ppm): 7.44 (d, J=4 Hz, 1 H), 6.94 (d, J=4 Hz, 1 H), 6.88-6.78 (m, 3 H), 5.25 (d, J=8 Hz, 1 H), 3.96-3.64 (m, 3 H), 3.3 (ddd, J=11, 4.6, 3.8 Hz, 1 H), 1.74 (t, J=4.6 Hz, 1 H). ¹⁹F NMR (CDCl₃, δ, ppm): −63.91 (s, 3 F), −108.07 (s, 2 F). MS (m/z, positive ESI, for M+H): 436.

### Example 3

### (S)-4-Benzyl-3-((S)-3-(3,5-difluorophenyl)-4,4,4-trifluorobutanoyl)oxazolidin-2-one

### A. (S,E)-4-Benzyl-3-(3-(3,5-difluorophenyl)-4,4,4-trifluorobut-2-enoyl)oxazolidin-2-one

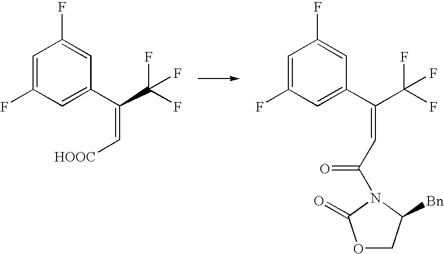

To a solution of (E)-3-(3,5-difluorophenyl)-4,4,4-trifluorobut-2-enoic acid (0.504 g, 2 mmol) in THF (3 mL) was added triethylamine (0.29 mL, 2.1 mmol, 1.05 equiv) at −7° C., followed by trimethylacetyl chloride (0.26 mL, 2.1 mmol, 1.05 equiv). The reaction mixture was stirred at −7 to −4° C. for 1.5 hours, then cooled to −78° C. In a separate flask, n-BuLi (2.2 mmol, 1.1 equiv., 0.88 mL of 2.5 M hexane solution) was added to a solution of (S)-(−)-4-benzyl-2-oxazolidinone (0.39 g, 2.2 mmol, 1.1 equiv.) in THF (3 mL) at −78° C. The mixture was stirred at −78° C. for 1.5 hours, added to the solution of mixed anhydride prepared above at −78° C.; chased with 5 mL of THF. The reaction mixture was stirred at −78° C. for 30 min, then warmed to room temperature, and stirred for 16 hours. 1N HCl was added at 0° C., followed by EtOAc. The phases were separated and the EtOAc phase was washed with Na₂CO₃ solution, dried over MgSO₄, and concentrated to afford 0.584 g of the title product (71% yield). ¹H NMR (CDCl₃, δ, ppm): 7.48 (q, J=1.5 Hz, 1 H), 7.36-7.23 (m, 3 H), 7.15-7.1 (m, 2 H), 6.94-6.85 (m, 3 H), 4.64-4.54 (m, 1 H), 4.28-4.16 (m, 2 H), 3.18 (dd, J=13.5, 3 Hz, 1 H), 2.67 (dd, J=13.5, 9.5 Hz, 1 H). ¹⁹F NMR (CDCl₃, δ, ppm): −67.68 (s, 3 F), −108.91 (s, 2 F). MS (m/z, positive ESI, for M+H): 412.

### B. (S)-4-Benzyl-3-((S)-3-(3,5-difluorophenyl)-4,4,4-trifluorobutanoyl)oxazolidin-2-one

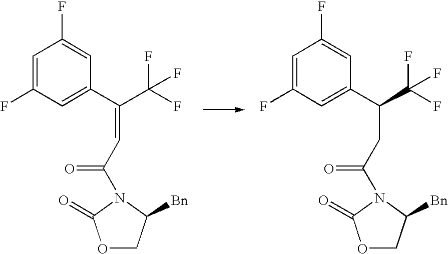

A mixture of (S,E)-4-benzyl-3-(3-(3,5-difluorophenyl)-4,4,4-trifluorobut-2-enoyl)oxazolidin-2-one (50 mg, 0.12 mmol), 10% Pd/C (6.5 mg, dry), MgBr₂ (22.4 mg, 0.12 mmol, 1 equiv.), and THF (2 mL) was hydrogenated at 450 psi H₂ and 50° C. for 24 hours. The mixture was filtered through a pad of the Celite® reagent, concentrated, redissolved in EtOAc, washed with 1N HCl, dried over MgSO₄, and concentrated to afford 38 mg of crude product. HPLC, ¹H NMR, ¹⁹F NMR analysis indicated formation of the title product as a major diastereomer (95:5 mixture of diastereomers; 21% of unreacted starting olefin remaining).

### Example 4

### (2S,3R)-2-Amino-3-(3,5-difluorophenyl)-4,4,4-trifluorobutan-1-ol hydrochloride

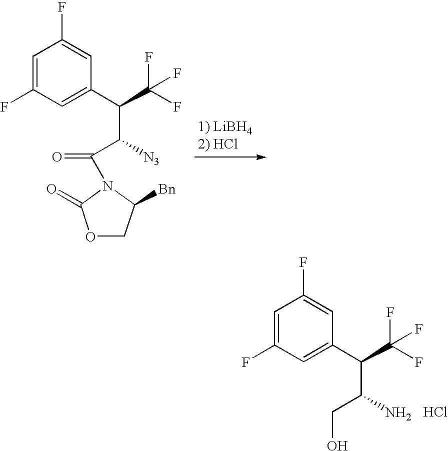

To a solution of (S)-3-((2S,3R)-2-azido-3-(3,5-difluorophenyl)-4,4,4-trifluorobutanoyl)-4-benzyloxazolidin-2-one (0.396 g of crude product of azidation) in THF (3 mL) was added LiBH₄ (2.6 mL of 2M THF solution) (exotherm). The reaction mixture was stirred at room temperature for 18 hours and then poured into MeOH. 2N HCl was added, the mixture was stirred at room temperature for 3 days, and concentrated in vacuum to remove THF and MeOH. Water, 2N HCl, and CH₂Cl₂ were added to the resultant suspension. The phases were separated and the aqueous phase was washed 2× with CH₂Cl₂, basified with K₂CO₃, and extracted 3× with MTBE. The combined MTBE fraction was dried over Na₂SO₄ and concentrated to afford 72 mg of crude product (free base) as colorless oil. Aqueous phase was saturated with NaCl, extracted additionally 3× with EtOAc. EtOAc fraction was dried over Na₂SO₄, and concentrated to afford 12 mg of crude product. Combined fractions of the crude product (free base) were dissolved in Et₂O (1 mL). MeOH (0.03 mL) was added, followed by 2N HCl solution in Et₂O (0.3 mL). The mixture was stirred at room temperature for 18 hours. The precipitate was filtered, washed with Et₂O and heptane to afford 79 mg of the title product. ¹H NMR (CD₃OD, δ, ppm): 7.15-7.05 (m, 3 H), 4.14-3.96 (m, 2 H), 3.64 (dd, J=12, 2 Hz, 1 H), 3.25 (dd, J=12, 3.5 Hz, 1 H). ¹⁹F NMR (CD₃OD, δ, ppm): −67.62 (s, 3 F), −110.9 (s, 2 F). MS (m/z, positive ESI, for M+H): 256.

### Example 5

### Analysis of 5-Chloro-N-((2S,3R)-3-(3,5-difluorophenyl)-4,4,4-trifluoro-1-hydroxybutan-2-yl)thiophene-2-sulfonamide

A sample of 5-Chloro-N-((2S,3R)-3-(3,5-difluorophenyl)-4,4,4-trifluoro-1-hydroxybutan-2-yl)thiophene-2-sulfonamide prepared according to Example [1 or 2] was analyzed using powder X-ray diffraction.

X-Ray diffraction data was acquired using a D8 ADVANCE® X-ray powder diffractometer (Bruker) having the following parameters and the X-ray diffraction pattern was obtained. See, FIG. 1.

| | |
|---|---|
| voltage: | 40 kV; |
| current: | 40.0 mA; |
| scan range (2θ): | 5 to 35°; |
| scan step size: | 0.01°; |
| total scan time: | 33 minutes; |
| detector: | VANTEC ™ detector; and |
| antiscattering slit: | 1 mm. |

All publications cited in this specification are incorporated herein by reference. While the invention has been described with reference to particular embodiments, it will be appreciated that modifications can be made without departing from the spirit of the invention. Such modifications are intended to fall within the scope of the appended claims.

What is claimed is:

1. A method for preparing a sulfonamide compound of the structure: 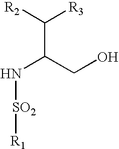
wherein:
R₁ is aryl, substituted aryl, heteroaryl, or substituted heteroaryl;
R₂ and R₃ are, independently, C₁ to C₆ alkyl, substituted C₁ to C₆ alkyl, aryl, substituted aryl, heteroaryl, and substituted heteroaryl;
said method selected from the group consisting of:
(a) a method comprising:
(i) enantioselectively hydrogenating R₂C(═CHCOOH)R₃ to R₂CH(CH₂COOH)R₃;
(ii) converting R₂CH(CH₂COOH)R₃ to an imide of a chiral oxazolidinone;
(iii) substituting the α-carbon atom of said imide of said chiral oxazolidinone with an azide;
(iv) converting said imide of said chiral oxazolidinone containing said azide to an amine or salt thereof;
(v) reducing said amine or salt thereof to an aminoalcohol or salt thereof; and
(vi) sulfonylating said aminoalcohol or salt thereof;
(b) a method comprising:
(i) enantioselectively hydrogenating R₂C(═CHCOOH)R₃ to R₂CH(CH₂COOH)R₃;
(ii) converting R₂CH(CH₂COOH)R₃ to an imide of a chiral oxazolidinone;
(iii) substituting the α-carbon atom of said imide of a chiral oxazolidinone with an azide;
(iv) converting said imide of a chiral oxazolidinone containing an azide to an imide of a chiral oxazolidinone containing an amine or salt thereof;
(v) sulfonylating said imide of a chiral oxazolidinone containing an amine or salt thereof; and
(vi) reducing said sulfonylated imide of a chiral oxazolidinone containing an amine or salt thereof;
(c) a method comprising:
(i) converting R₂C(═CHCOOH)R₃ to an unsaturated imide of a chiral oxazolidinone;
(ii) diastereoselectively hydrogenating said unsaturated imide of a chiral oxazolidinone to an imide of a chiral oxazolidinone;
(iii) substituting the α-carbon atom of said imide of said chiral oxazolidinone with an azide;
(iv) converting said imide of said chiral oxazolidinone with said azide to an amine or salt thereof;
(v) reducing said amine or salt thereof to an aminoalcohol or salt thereof; and
(vi) sulfonylating said aminoalcohol or salt thereof;
(d) a method comprising:
(i) enantioselectively hydrogenating R₂C(═CHCOOH)R₃ to R₂CH(CH₂COOH)R₃;
(ii) converting R₂CH(CH₂COOH)R₃ to an imide of a chiral oxazolidinone;
(iii) substituting the α-carbon atom of said imide of said chiral oxazolidinone with an azide;
(iv) converting said imide of said chiral oxazolidinone containing said azide to an aminoalcohol or salt thereof; and
(v) sulfonylating said aminoalcohol or salt thereof;
(e) a method comprising:
(i) converting R₂C(═CHCOOH)R₃ to an unsaturated imide of a chiral oxazolidinone;
(ii) diastereoselectively hydrogenating said unsaturated imide of a chiral oxazolidinone to an imide of a chiral oxazolidinone;
(iii) substituting the α-carbon atom of said imide of said chiral oxazolidinone with an azide;
(iv) converting said imide of said chiral oxazolidinone with said azide to an aminoalcohol or salt thereof; and
(v) sulfonylating said aminoalcohol or salt thereof; and
(f) a method comprising:
(i) converting R₂C(═CHCOOH)R₃ to an unsaturated imide of a chiral oxazolidinone;
(ii) diastereoselectively hydrogenating said unsaturated imide of a chiral oxazolidinone to an imide of a chiral oxazolidinone;
(iii) substituting the α-carbon atom of said imide of a chiral oxazolidinone with an azide;
(iv) converting said imide of a chiral oxazolidinone containing an azide to an imide of a chiral oxazolidinone containing an amine or salt thereof;
(v) sulfonylating said imide of a chiral oxazolidinone containing an amine or salt thereof; and
(vi) reducing said imide of a chiral oxazolidinone containing an amine or salt thereof.

2. The method according to claim 1, which is one of methods (a)-(e) and wherein R₂C(═CHCOOH)R₃ is (E)-3-(3,5-difluorophenyl)-4,4,4-trifluoro-but-2-enoic acid.

3. The method according to claim 1 which is one of methods (a), (b), or (d) and wherein R₂CH(CH₂COOH)R₃ is (S)-3-(3,5-difluorophenyl)-4,4,4-trifluorobutanoic acid.

4. The method according to claim 1, wherein step (iii) is stereoselective.

5. The method according to claim 1, wherein said sulfonylation is performed using a compound of the structure: 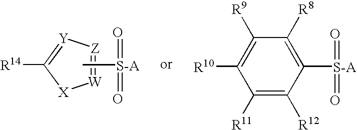
wherein:
A is a leaving group;
R¹⁴ is selected from the group consisting of H, halogen, and CF₃;
W, Y and Z are independently selected from the group consisting of C, CR⁶ and N, wherein at least one of W, Y or Z is C;
X is selected from the group consisting of O, S, SO₂, and NR⁷;
R⁶ is selected from the group consisting of H, halogen, C₁ to C₆ alkyl, and substituted C₁ to C₆ alkyl;
R⁷ is selected from the group consisting of H, C₁ to C₆ alkyl, C₃ to C₈ cycloalkyl, SO₂(C₁ to C₆ alkyl), SO₂(substituted C₁ to C₆ alkyl), SO₂aryl, SO₂substituted aryl, CO(C₁ to C₆ alkyl), CO(substituted C₁ to C₆ alkyl), COaryl and COsubstituted aryl;
R⁸, R⁹, R¹⁰, R¹¹, and R¹² are independently selected from the group consisting of H, halogen, C₁ to C₆ alkoxy, substituted C₁ to C₆ alkoxy, NO₂, C₁ to C₆ alkyl, substituted C₁ to C₆ alkyl, CN, C₁ to C₆ alkylcarbonyl, substituted C₁ to C₆ alkylcarbonyl, C₁ to C₆ alkylcarboxy, substituted C₁ to C₆ alkylcarboxy, CONH₂, CONH(C₁ to C₆ alkyl), CONH(substituted C₁ to C₆ alkyl), CON(C₁ to C₆ alkyl)₂, CON(substituted C₁ to C₆ alkyl)₂, S(C₁ to C₆ alkyl), S(substituted C₁ to C₆ alkyl), SO(C₁ to C₆ alkyl), SO(substituted C₁ to C₆ alkyl), SO₂(C₁ to C₆ alkyl), SO₂(substituted C₁ to C₆ alkyl), NHSO₂(C₁ to C₆ alkyl), and NHSO₂(substituted C₁ to C₆ alkyl); or
R⁸ and R⁹; R⁹ and R¹⁰; R¹¹ and R¹²; or R¹⁰ and R¹¹ are fused to form:
(i) a carbon-based saturated ring containing 3 to 8 carbon atoms;
(ii) a carbon-based unsaturated ring containing 3 to 8 carbon atoms; or
(iii) a heterocyclic ring containing 1 to 3 heteroatoms selected from the group consisting of O, N, and S in the backbone of the ring;
wherein rings (i) to (iii) may be substituted by 1 to 3 substituents including C₁ to C₆ alkyl or substituted C₁ to C₆ alkyl.

6. The method according to claim 1 which is one of methods (a) to (e) and wherein the product of step (ii) is of the structure: 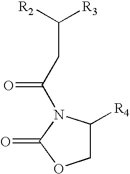
wherein, R₄ is C₁ to C₆ alkyl, substituted C₁ to C₆ alkyl, aryl, or substituted aryl.

7. The method according to claim 6, wherein the product of step (ii) is (S)-4-Benzyl-3-((S)-3-(3,5-difluorophenyl)-4,4,4-trifluorobutanoyl)oxazolidin-2-one.

8. The method according to claim 1 which is one of methods (a) to (e) and wherein the product of step (iii) is of the structure: 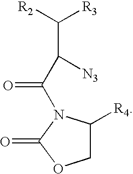
wherein, R₄ is C₁ to C₆ alkyl, substituted C₁ to C₆ alkyl, aryl, or substituted aryl.

9. The method according to claim 8, wherein the product of step (iii) is (S)-3-((2S,3R)-2-Azido-3-(3,5-difluorophenyl)-4,4,4-trifluorobutanoyl)-4-benzyloxazolidin-2-one.

10. The method according to claim 1 which is one of methods (a)-(c) or (e) and wherein said amine or salt thereof is of the structure: 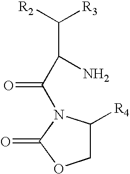
of a salt thereof, wherein R₄ is C₁ to C₆ alkyl, substituted C₁ to C₆ alkyl, aryl, or substituted aryl.

11. The method according to claim 10, wherein said amine salt is (S)-3-((2S-3R)-2-Amino-3-(3,5-difluorophenyl)-4,4,4-trifluorobutanoyl)-4-benzyloxazolidin-2-one hydrochloride.

12. The method according to claim 1 which is one of methods (a) or (c)-(e) and wherein said aminoalcohol salt is of the structure:

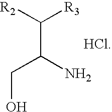

13. The method according to claim 12, wherein said aminoalcohol salt is (2S,3R)-2-Amino-3-(3,5-difluorophenyl)-4,4,4-trifluorobutan-1-ol hydrochloride.

14. The method according to claim 1, wherein said sulfonamide compound is of the structure:

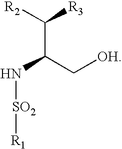

15. The method according to claim 1, wherein said sulfonamide compound is 5-Chloro-N-((2S,3R)-3-(3,5-difluorophenyl)-4,4,4-trifluoro-1-hydroxybutan-2-yl)thiophene-2-sulfonamide.

16. The method according to claim 15, wherein the powder X-ray diffraction pattern of 5-Chloro-N-((2S,3R)-3-(3,5-difluorophenyl)-4,4,4-trifluoro-1-hydroxybutan-2-yl)thiophene-2-sulfonamide comprises a peak at 2° of about 6.7°±0.3°.

17. The method according to claim 16, wherein the powder X-ray diffraction pattern further comprises one or more peaks at 2θ of about 15.1°±0.3°, 15.0°±0.3°, 16.3°±0.3°, 17.8°±0.3°, 18.4°±0.3°, 19.7°±0.3°, 21.1°±0.3°, 22.2°±0.3°, 22.7°±0.3°, 23.4°±0.3°, or 24.5°±0.3°.

18. The method according to claim 1 which is method (b) or (e) and wherein the product of step (v) is N-((2S,3R)-1-((S)-4-Benzyl-2-oxo-oxazolidin-3-yl)-3-(3,5-difluorophenyl)-4,4,4-trifluoro-1-oxobutan-2-yl)-5-chlorothiophene-2-sulfonamide.

19. The method according to claim 1 which is method (a), (b), or (d) and wherein R₂CH(CH₂COOH)R₃ is present at greater than 95% enantiomeric excess.

20. The method according to claim 1 which is method (c), (e), or (f) and wherein the product of step (i) is of the structure: 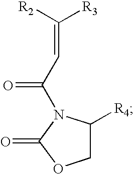
wherein, R₄ is C₁ to C₆ alkyl, substituted C₁ to C₆ alkyl, aryl, or substituted aryl.

21. The method according to claim 20, wherein the product of step (i) is (S,E)-4-benzyl-3-(3-(3,5-difluorophenyl)-4,4,4-trifluorobut-2-enoyl)oxazolidin-2-one.

22. The method according to claim 1, wherein the sulfonamide compound is purified and wherein the purification of the sulfonamide is performed in the absence of chromatographic separation of isomers.

23. A method for enantioselectively preparing a chiral compound, or derivative thereof, of the structure: 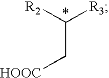
wherein, R₂ and R₃ are, independently, C₁ to C₆ alkyl, substituted C₁ to C₆ alkyl, aryl, substituted aryl, heteroaryl, and substituted heteroaryl;
said method comprising:
(i) converting R₂C(═CHCOOH)R₃ to a chiral oxazolidinone of the structure: 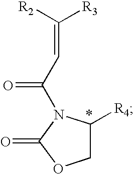
wherein, R₄ is C₁ to C₆ alkyl, substituted C₁ to C₆ alkyl, aryl, or substituted aryl;
(ii) hydrogenating the product of step (i); and
(iii) converting the product of step (ii) to said chiral compound.

24. The method according to claim 23, wherein said chiral compound is:

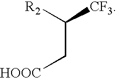

25. The method according to claim 23, wherein said chiral compound is:

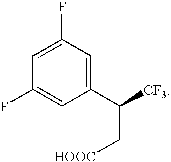

26. A compound which is selected from the group consisting of (a) (S)-3-(3,5-difluorophenyl)-4,4,4-trifluorobutanoic acid, (b) (S)-4-Benzyl-3-((S)-3-(3,5-difluorophenyl)-4,4,4-trifluorobutanoyl)oxazolidin-2-one, (c) (S)-3-((2S,3R)-2-Azido-3-(3,5-difluorophenyl)-4,4,4-trifluorobutanoyl)-4-benzyloxazolidin-2-one, (d) (S)-3-((2S,3R)-2-Amino-3-(3,5-difluorophenyl)-4,4,4-trifluorobutanoyl)-4-benzyloxazolidin-2-one hydrochloride, (e) (2S,3R)-2-Amino-3-(3,5-difluorophenyl)-4,4,4-trifluorobutan-1-ol hydrochloride, and (f) N-((2S,3R)-1-((S)-4-Benzyl-2-oxo-oxazolidin-3-yl)-3-(3,5-difluorophenyl)-4,4,4-trifluoro-1-oxobutan-2-yl)-5-chlorothiophene-2-sulfonamide.

27. A method for preparing compound (a) of claim 26, said method comprising hydrogenating (E)-3-(3,-Difluorophenyl)-4,4,4-trifluorobut-2-enoic acid using a transition metal catalyst comprising chiral non-racemic ligands and hydrogen.

28. A method for preparing compound (b) of claim 26, said method comprising:
(I) a method comprising:
(i) reacting (S)-3-(3,5-difluorophenyl)-4,4,4-trifluorobutanoic acid, triethylamine, and pivaloyl chloride; and
(ii) reacting the product of step (i) with lithium (S)-(−)-4-benzyl-2-oxazolidinone;
(II) a method comprising:
(i) reacting (S)-3-(3,5-difluorophenyl)-4,4,4-trifluorobutanoic acid and oxalyl chloride; and
(ii) reacting the product of step (i) with (S)-(−)-4-benzyl-2-oxazolidinone or a salt thereof;
(III) a method comprising:
(i) reacting (S)-3-(3,5-difluorophenyl)-4,4,4-trifluorobutanoic acid, triethylamine, and pivaloyl chloride; and
(ii) reacting the product of step (i) with (S)-(−)-4-benzyl-2-oxazolidinone and a base; or
(IV) a method comprising:
(i) reacting (S)-3-(3,5-difluorophenyl)-4,4,4-trifluorobutanoic acid and oxalyl chloride; and
(ii) reacting the product of step (i) with a Lewis acid.

29. A method for preparing compound (c) of claim 24, said method comprising reacting (S)-4-Benzyl-3-((S)-3-(3,5-difluorophenyl)-4,4,4-trifluorobutanoyl)oxazolidin-2-one, potassium hexamethyldisilazide and 2,4,6-triisopropylsulfonylazide.

30. A method for preparing compound (d) of claim 24, said method comprising hydrogenating (S)-3-((2S,3R)-2-Azido-3-(3,5-difluorophenyl)-4,4,4-trifluorobutanoyl)-4-benzyloxazolidin-2-one in the presence of hydrochloric acid.

31. A method for preparing compound (e) of claim 26, comprising:
(I) a method comprising:
(i) reacting (S)-3-((2S,3R)-2-Amino-3-(3,5-difluorophenyl)-4,4,4-trifluorobutanoyl)-4-benzyloxazolidin-2-one hydrochloride and lithium borohydride; and
(ii) reacting the product of step (i) with hydrochloric acid; or
(II) a method comprising:
(i) reacting (S)-3-((2S,3R)-2-Azido-3-(3,5-difluorophenyl)-4,4,4-trifluorobutanoyl)-4-benzyloxazolidin-2-one and lithiumborohydride; and
(ii) reacting the product of step (i) with hydrochloric acid.

32. A method for preparing compound (f) of claim 26, said method comprising reacting (S)-3-((2S,3R)-2-Amino-3-(3,5-difluorophenyl)-4,4,4-trifluorobutanoyl)-4-benzyloxazolidin-2-one hydrochloride, a pyridine compound, and 5-chlorothiophene-2-sulfonyl chloride.
